SEQUENCE LISTING (7 sequences)

SEQ ID NO: 1 - protein, 436 aa, Artificial Sequence
  Description of Artificial Sequence Synthetic polypeptide
  alpha-amylase
MAKYSELEKGGVIMQAFYWDVPSGGIWWDTIRQKIPEWYDAGISAIWIPPASKGMGGAYSMGYDPYDFFDLGEYDQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNKNIPALVSALQNGQTVVSRDPFKAVTFVANHDTDIIWNKYPAYAFILTYEGQPTIFYRDYEEWLNKDKLKNLIWIHENLAGGSTDIVYYDNDELIFVRNGYGDKPGLITYINLGSSKAGRWVYVPKFAGACIHEYTGNLGGWVDKYVYSSGWVYLEAPAYDPANGQYGYSVWSYCGVG

SEQ ID NO: 2 - protein, 549 aa, Geobacillus stearothermophilus
MLTFHRIIRKGWMFLLAFLLTALLFCPTGQPAKAAAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAWP

SEQ ID NO: 3 - protein, 436 aa, Artificial Sequence
  Description of Artificial Sequence Synthetic polypeptide
  amylase backbone
MAKYSELEEGGVIMQAFYWDVPGGGIWWDTIRSKIPEWYEAGISAIWIPPASKGMSGGYSMGYDPYDFFDLGEYNQKGTIETRFGSKQELINMINTAHAYGIKVIADIVINHRAGGDLEWNPFVGDYTWTDFSKVASGKYTANYLDFHPNEVKCCDEGTFGGFPDIAHEKSWDQHWLWASDESYAAYLRSIGVDAWRFDYVKGYGAWVVKDWLNWWGGWAVGEYWDTNVDALLNWAYSSGAKVFDFPLYYKMDEAFDNKNIPALVSALQNGQTVVSRDPFKAVTFVANHDTDIIWNKYLAYAFILTYEGQPVIFYRDYEEWLNKDRLNNLIWIHDHLAGGSTSIVYYDSDEMIFVRNGYGSKPGLITYINLGSSKVGRWVYVPKFAGACIHEYTGNLGGWVDKYVYSSGWVYLEAPAYDPANGQYGYSVWSYCGVG

SEQ ID NO: 4 - protein, 436 aa, Artificial Sequence
  Description of Artificial Sequence Synthetic polypeptide
  amylase backbone
MAKYLELEEGGLIMQAFYWDVPMGGIWWDTIAQKIPDWASAGISAIWIPPASKGMSGGYSMGYDPYDYFDLGEYYQKGTVETRFGSKQELINMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNNNIPALVDALRYGQTVVSRDPFKAVTFVANHDTDIIWNKYPAYAFILTYEGQPTIFYRDYEEWLNKDKLKNLIWIHDNLAGGSTDIVYYDNDELIFVRNGYGSKPGLITYINLASSKAGRWVYVPKFAGSCIHEYTGNLGGWVDKWVDSSGWVYLEAPAHDPANGQYGYSVWSYCGVG

SEQ ID NO: 5 - protein, 436 aa, Artificial Sequence
  Description of Artificial Sequence Synthetic polypeptide
  amylase backbone
MAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 6 - protein, 436 aa, Artificial Sequence
  Description of Artificial Sequence Synthetic polypeptide
  amylase backbone
MAKYSELEKGGVIMQAFYWDVPEGGIWWDTIRQKIPEWYDAGISAIWIPPASKGMGGAYSMGYDPYDFFDLGEYDQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASQESYAAYLRSIGIDAWRFDYVKGYAPWVVKDWLNWWGGWAVGEYWDTNVDAVLNWAYSSGAKVFDFALYYKMDEAFDNKNIPALVSALQNGQTVVSRDPFKAVTFVANHDTDIIWNKYPAYAFILTYEGQPTIFYRDYEEWLNKDKLKNLIWIHENLAGGSTDIVYYDNDELIFVRNGYGDKPGLITYINLGSSKAGRWVYVPKFAGACIHEYTGNLGGWVDKYVYSSGWVYLEAPAYDPANGQYGYSVWSYCGVG

SEQ ID NO: 7 - protein, 436 aa, Artificial Sequence
  Description of Artificial Sequence Synthetic polypeptide
  amylase backbone
MAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

(19) United States
(12) Patent Application Publication
(10) Pub. No.: US 2025/0340913 A1
(43) Pub. Date: Nov. 6, 2025

(54) METHOD OF STARCH PROCESSING

(71) Applicant: DANSTAR FERMENT AG, ZUG (CH)

(72) Inventors: Anthony Newton, San Diego, CA (US); Amanda Huber, San Diego, CA (US); Yukiko Sato, San Diego, CA (US); Asfia Qureshi, San Diego, CA (US)

(21) Appl. No.: 18/266,938

(22) PCT Filed: Dec. 14, 2021
(86) PCT No.: PCT/US2021/063344

§ 371 Date: Jun. 13, 2023

Related U.S. Application Data
(60) Provisional application No. 63/125,039, filed on Dec. 14, 2020.

(51) Int. Cl. C12P 19/02; C12N 9/28; C12P 19/16

(52) U.S. Cl. CPC C12P 19/02; C12N 9/2417; C12P 19/16; C12Y 302/01001

(57) ABSTRACT

The present application relates to methods of starch processing and the production of fermentation products, such as ethanol, the methods comprising the addition of a first and a second alpha-amylase and optionally the addition of proteases, glucoamylases and other enzymes.

### SEQUENCE LISTING

The instant application contains a Sequence Listing which has been submitted electronically in ASCII format and is hereby incorporated by reference in its entirety. Said ASCII copy, created on Jun. 28, 2023, is named 191315US02 BAF-23-1145US SL.txt and is 28,426 bytes in size.

### FIELD OF THE INVENTION

The present application relates to methods of starch processing and the production of fermentation products, such as ethanol, the methods comprising the addition of a first and a second alpha-amylase and optionally the addition of proteases, glucoamylases and other enzymes.

### BACKGROUND OF THE INVENTION

Starch processing is an industrial process by which starch derived from raw plant material is broken down into sugars. Sugars and by-products of the process are further processed into products such as alcohol, oil, syrup, animal feed, biofuels, food and beverage items.

To produce ethanol, starch-containing fractions derived from wet milling or ground grain from dry grinding are further hydrolyzed into fermentable sugars which are then fermented to make ethanol. Several plant starch-processing methods exist including high temperature hydrolysis of starch, frequently referred to as “liquefaction”. Methods for breaking down starch derived from plants conventionally involve the addition of enzymes, frequently liquid enzymes, to the milled plant starch in a slurry tank.

Liquefaction methods often involve a starch gelatinization process, wherein aqueous starch slurry is heated so that the granular starch in the slurry swells and bursts, dispersing starch molecules into the solution. During the gelatinization process, there is a dramatic increase in viscosity. To enable handling during the remaining process steps, the starch must be “liquefied” to reduce the viscosity. This reduction in viscosity can be accomplished by enzymatic degradation during liquefaction. The long-chained starch molecules are degraded into smaller branched and linear chains of glucose units (dextrins) by one or more enzymes, such as alpha-amylase.

Amylases are enzymes that catalyse the hydrolysis of starch into sugars. As glycoside hydrolases, amylases act on alpha-1,4-glycosidic bonds.

Alpha-amylases (1,4-alpha-D-glucan glucanohydrolase, E.C. 3.2.1.1) constitute a group of enzymes which act on starch, glycogen and related polysaccharides and oligosaccharides in a random manner, by catalyzing the hydrolysis of (1→4)-alpha-D-glucosidic linkages in polysaccharides containing three or more (1→4)-alpha-linked D-glucose units. For example, alpha-amylases hydrolyse amylose and amylopectin, the polysaccharides found in starch, breaking them down into polysaccharide, oligosaccharide or glucose.

Alpha-amylases can be used commercially in the initial stages of starch processing; in wet corn milling; in alcohol production, for example in fermentation; as cleaning agents such as clothing and dishwasher detergents; in the textile industry for starch desizing; in baking applications to break down complex sugars found in flour; in the beverage industry; in oilfields in drilling processes; in deinking of recycled paper and as a food additive.

Alpha-amylases can be isolated from a wide variety of bacterial, fungal, plant, and animal sources. Many industrially important alpha-amylases are isolated from Bacillus sp., in part because of the generally high capacity of Bacillus to secrete amylases into the growth medium. Furthermore, there is a need for blends of alpha-amylases, or variants thereof, which can capitalize on the best properties of at least two alpha-amylases from at least two bacterial strains.

For example, alpha-amylases isolated from Bacillus stearothermophilus have been used in fuel ethanol applications because of their rapid viscosity decreasing property. However, certain alpha-amylases or variants thereof are not thermostable, so while they decrease the viscosity of a slurry over time, they suffer from lower viscosity reduction in secondary liquefaction, where the slurry may be kept at 85-90° C. for up to 60-210 minutes.

EP 0 252 730 A2 discloses an enzyme product comprising a mixture of an alpha-amylase from Bacillus licheniformis and an alpha-amylase from Bacillus stearothermophilus, said mixture containing from 10-90% by activity of the Bacillus licheniformis enzyme. The amylase mixture is used for liquefaction of starch or starchy grains.

U.S. Pat. No. 4,933,279 A discloses a mixed enzyme product comprising a mixture of an alpha-amylase from Bacillus licheniformis and an alpha-amylase from B. stearothermophilus, wherein said mixture contains from 10%-90%, preferably 25%-90%, more preferably 25%-75% by activity as NU/g DS of the Bacillus licheniformis enzyme and is usable for liquefaction of starch or starchy grains.

WO 2005/086640 A2 discloses a process of liquefying starch-containing material comprising the step of treating said starch-containing material with at least one alpha-amylase and a maltogenic amylase or at least one amylase and at least one esterase.

WO 2009/052101 A1 discloses an enzyme blend composition comprising a glucoamylase, an acid stable alpha amylase, and an acid fungal protease

WO 2010/036515 A1 discloses a blend of a Geobacillus stearothermophilus alpha-amylase and a Bacillus licheniformis alpha-amylase.

WO 2011/017093 A1 discloses an enzyme blend for processing a starch comprising a low pH, thermostable alpha-amylase and a Bacillus licheniformis alpha-amylase.

U.S. Pat. No. 10,689,679 B2 discloses a process for starch liquefaction using at least two classes of α-amylase enzymes, wherein the starch hydrolysis pattern from at least two of these classes is different.

Nevertheless, there is a need in the industry for the identification and optimization of alpha-amylase blends which are useful in various production processes, for example, commercial starch liquefaction processes and ethanol production processes. For example, there remains a need to improve the viscosity of the slurry, given its significant impact on downstream processes and the yield and/or quality of end products. Low viscosity starch liquefacts are useful in the current ethanol production process. If a way could be found to produce such low viscosity liquefacts as fermentation feedstocks using an optimized blend of alpha-amylases, or variants thereof, this would represent a useful contribution to the art. Furthermore, if a way could be found to treat whole ground grains with a blend of alpha-amylases, or variants thereof, to improve starch liquefaction, this would also represent a useful contribution to the art. Additionally, enzyme blends comprising alpha-amylases, proteases and glucoamylases for starch processing to obtain products such as ethanol and corn oil that improve yields of those products would be a useful contribution to the art.

### SUMMARY OF THE INVENTION

The present invention provides improved methods for starch processing using a first alpha-amylase together with a second alpha-amylase and optionally using a first alpha-amylase together with a second alpha-amylase and a protease. Improved viscosity, ethanol yield and corn oil extraction were obtained using the methods of the invention.

Accordingly, the invention relates to a method of starch processing, comprising the steps of:
(a) providing a first alpha-amylase according to SEQ ID NO: 1 or a variant thereof having an amino acid sequence which is at least 80% identical to the amino acid sequence according to SEQ ID NO: 1;
(b) providing a second alpha-amylase;
(c) adding (a) and (b) to a slurry comprising a starch, thereby forming a mixture and incubating said mixture.

In one embodiment, said variant of the first alpha-amylase comprises at least one amino acid modification at an amino acid residue position number selected from the group consisting of: 23, 33, 181, 260, 272, 323, 349, 357, 407, and 408 or a combination thereof in the numbering of any one of SEQ ID NOs: 1, 3, 4, 5, 6 and 7.

In one embodiment, said amino acid modification(s) is/are an amino acid substitution, insertion, deletion, or any combination thereof.

In one embodiment, said amino acid modification(s) is/are an amino acid substitution, and wherein the amino acid substitution is a conservative amino acid substitution.

In one embodiment, said at least one amino acid modification is an amino acid substitution selected from the group consisting of: 23E, 33E, 181E, 260D/E, 272D/E, 323E, 349P, 357E, 407E and 408E or a combination thereof in the numbering of any one of SEQ ID Nos. 1, 3, 4, 5, 6 and 7.

In one embodiment, said variant of the first alpha-amylase comprises the amino acid modifications of:
(a) 260D, or
(b) 357E, or
(c) 407E
(d) 408E, or
(e) 23E, 33E, 181E, 260E, 272D, 323E, 349P, 357E, and 407E, or
(f) 23E, 260E, 272E, and 407E.

In one embodiment, said mixture is incubated for a period of about 10 minutes to about 60 minutes.

In one embodiment, the method of starch processing further comprises adding (a) and (b) during a liquefying stage.

In one embodiment, said mixture is incubated for a period of about 60 minutes to about 210 minutes during the liquefying stage.

In one embodiment, the method is performed at a temperature of about 62° C. to about 95° C.

In one embodiment, the method is performed at a pH of 4.3 to 6.5.

In one embodiment, the first alpha-amylase of (a) and the second alpha-amylase of (b) are added simultaneously and/or separately.

In one embodiment, the second alpha-amylase is an alpha-amylase from Geobacillus stearothermophilus or a variant thereof.

In one embodiment, the starch is derived from raw plant material and the first alpha-amylase (a) is present in an amount of from about 0.001% to about 0.05% weight of enzyme by weight of raw plant material.

In one embodiment, the second alpha-amylase (b) is present at 1% to 20% inclusion rate, preferably at 10% to 20% inclusion rate.

In one embodiment, the weight ratio of the first alpha-amylase to the second alpha-amylase is between about 20:1 to about 1:10.

In one embodiment, the starch is derived from raw plant material and the total enzyme dose is between about 0.002% to about 0.05% total weight of enzyme by weight of raw plant material.

In one embodiment, the viscosity of the slurry is reduced by at least 10% compared to a slurry not comprising the second alpha-amylase of (b).

In one embodiment, the slurry comprises 20% to 40% solids.

In one embodiment, the method of starch processing further comprises saccharifying and fermenting the slurry of step (c) to produce a fermentation product comprising alcohols.

In one embodiment, the method of starch processing further comprises recovering the fermentation product.

In one such embodiment, the fermentation product is ethanol.

In one embodiment, the method further comprises distilling the fermentation product to produce ethanol and whole stillage, wherein the whole stillage is processed to produce one or more of wet distiller's grains with solubles (WDGS) and dried distiller's grains with solubles (DDGS).

In one embodiment, the starch is derived from corn and the whole stillage is processed to produce corn oil.

In some embodiments of the method of starch processing, in step (c) a composition comprising the second alpha-amylase and a first protease is added.

In one such embodiment, the first alpha-amylase and the composition comprising the second alpha-amylase and the first protease are added simultaneously and/or separately.

In one such embodiment, the starch is derived from raw plant material and the first alpha-amylase is added at an amount of about 0.01% to about 0.06% weight of enzyme per weight of raw plant material.

In one such embodiment, the starch is derived from raw plant material and the composition comprising the second alpha-amylase and the first protease is added at an amount of about 0.001% to about 0.01% weight of enzyme per weight of raw plant material.

In one such embodiment, the method of starch processing further comprises adding a second protease and/or a glucoamylase.

In one embodiment, the method of starch processing, comprising adding the first alpha-amylase and a composition comprising second amylase and the first protease, further comprises saccharifying and fermenting the slurry of step (c) to produce a fermentation product comprising alcohols.

In one such embodiment, the method of starch processing further comprises recovering the fermentation product.

In one such embodiment, the fermentation product is ethanol.

In one such embodiment, the ethanol yield is increased by 0.5% to 5% compared to a slurry not comprising the first alpha-amylase.

In one such embodiment, the method further comprises distilling the fermentation product to produce ethanol and whole stillage, wherein the whole stillage is processed to produce one or more of wet distiller's grains with solubles (WDGS) and dried distiller's grains with solubles (DDGS).

In one such embodiment, the starch is derived from corn and the whole stillage is processed to produce corn oil.

In one such embodiment, the corn oil extraction yield is increased by 4% to 12% compared to a slurry not comprising the first alpha-amylase.

In one embodiment a composition comprising a variant of a first alpha-amylase and a second alpha-amylase is disclosed. The variant of the first alpha-amylase has an amino acid sequence which is at least 80% identical to the amino acid sequence according to any one of SEQ ID NOs: 1, 3, 4, 5, 6 and 7 and which comprises at least one amino acid modification at an amino acid residue position number selected from the group consisting of: 23, 33, 181, 260, 272, 323, 349, 357, 407, and 408 or a combination thereof in the numbering of any one of SEQ ID Nos. 1, 3, 4, 5, 6 and 7. In a preferred embodiment, the at least one amino acid modification is an amino acid substitution selected from the group consisting of: 23E, 33E, 181E, 260D/E, 272D/E, 323E, 349P, 357E, 407E and 408E or a combination thereof in the numbering of any one of SEQ ID Nos. 1, 3, 4, 5, 6 and 7. In a preferred embodiment the variant of the first alpha-amylase comprises the amino acid modifications of: (a) 260D, or 357E, or 407E, 408E, or 23E, 33E, 181E, 260E, 272D, 323E, 349P, 357E, and 407E, or 23E, 260E, 272E, and 407E. In a preferred embodiment, the second alpha-amylase is an alpha-amylase from Geobacillus stearothermophilus or a variant thereof. In one embodiment the composition further comprises a protease and/or glucoamylase.

### BRIEF DESCRIPTION OF THE DRAWINGS

FIG. 1A: Viscosity measurements comparing incubation of slurry mixtures with the first alpha-amylase according to SEQ ID NO: 1, the second alpha-amylase (SUKAMY HI from Shandong Sukahan Bio-Technology) or different ratios of both the first and the second alpha-amylase at 80° C. and at 90° C.

FIG. 1B: Viscosity measurements comparing incubation of slurry mixtures comprising 32% solids with the first alpha-amylase according to SEQ ID NO: 1 and the second alpha-amylase (HTAA180L from Sunson) at inclusion rates of 0%, 5%, 10%, 15% and 20%.

FIG. 1C: Viscosity measurements comparing incubation of slurry mixtures comprising 35% solids with the first alpha-amylase according to SEQ ID NO: 1 and the second alpha-amylase (HTAA180L from Sunson) at inclusion rates of 0%, 5%, 10%, 15% and 20%.

FIG. 2A: Ethanol yield of starch processing, comparing incubation of a slurry with the first alpha-amylase according to SEQ ID NO: 1 (AA2) and Avantec® Amp from Novozymes and a slurry comprising Liquozyme® SC DS (AA1) and Avantec® Amp from Novozymes.

FIG. 2B: Corn oil yield of a method of starch processing, comparing a method comprising incubating a slurry comprising the first alpha-amylase according to SEQ ID NO: 1 (AA2) and Avantec® Amp from Novozymes with a method comprising incubating a slurry comprising Liquozyme® SC DS (AA1) and Avantec® Amp from Novozymes.

FIG. 3: Viscosity measurements comparing incubation of slurry mixtures comprising 35% solids with Variant 4 and the second alpha-amylase (HTAA180L from Sunson) at inclusion rates of 0%, 5%, 10%, 15% and 20%. Condition 2 was not determined. Blend conditions are shown in Table 8.

FIG. 4: Viscosity measurements comparing incubation of slurry mixtures comprising 35% solids with Variant 5 and the second alpha-amylase (HTAA180L from Sunson) at inclusion rates of 0%, 5%, 10%, 15% and 20%. Condition 2 was not determined. Blend conditions are shown in Table 8.

FIG. 5: Viscosity measurements comparing incubation of slurry mixtures comprising 35% solids with Variant 7 and the second alpha-amylase (HTAA180L from Sunson) at inclusion rates of 0%, 5%, 10%, 15% and 20%. Blend conditions are shown in Table 10.

FIG. 6: Viscosity measurements comparing incubation of slurry mixtures comprising 35% solids with Variant 8 and the second alpha-amylase (HTAA180L from Sunson) at inclusion rates of 0%, 5%, 10%, 15% and 20%. Blend conditions are shown in Table 10.

FIG. 7: Viscosity measurements comparing incubation of slurry mixtures comprising 35% solids with Variant 12 and the second alpha-amylase (HTAA180L from Sunson) at inclusion rates of 0%, 5%, 10%, 15% and 20%. Blend conditions are shown in Table 9.

FIG. 8: Viscosity measurements comparing incubation of slurry mixtures comprising 35% solids with Variant 13 and the second alpha-amylase (HTAA180L from Sunson) at inclusion rates of 0%, 5%, 10%, 15% and 20%. Blend conditions are shown in Table 9.

FIG. 9: Viscosity measurements comparing incubation of slurry mixtures comprising 35% solids with Variant 17 and the second alpha-amylase (HTAA180L from Sunson) at inclusion rates of 0%, 5%, 10%, 15% and 20%. Blend conditions are shown in Table 10.

FIG. 10: Viscosity measurements comparing incubation of slurry mixtures comprising 35% solids with Variant 18 and the second alpha-amylase (HTAA180L from Sunson) at inclusion rates of 0%, 5%, 10%, 15% and 20%. Blend conditions are shown in Table 10.

FIG. 11: Viscosity measurements comparing incubation of slurry mixtures comprising 35% solids with Variant 24 and the second alpha-amylase (HTAA180L from Sunson) at inclusion rates of 0%, 5%, 10%, 15% and 20%. Blend conditions are shown in Table 11.

### DETAILED DESCRIPTION OF THE INVENTION

### Definitions

Although the present invention will be described with respect to particular embodiments, this description is not to be construed in a limiting sense.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

### Starch and Starch Slurry

As described above, the present invention provides improved methods of starch processing.

The term “starch processing” refers to the industrial process of break-down of the starch into sugars and, optionally, the chemical and mechanical conversion of these sugars into the desired products such as ethanol, oil, syrup, animal feed, biofuels, food and beverage items. Plants are commonly used as an industrial source for starch. Plant starches are generally in a granular form, which is insoluble in water.

As used herein the term “starch” refers to any material composed of amylose and amylopectin. Amylose is a polysaccharide made of glucose units, bonded to each other through α(1→4) glycosidic bonds. Amylopectin is a water-soluble polysaccharide and highly branched polymer of glucose units. In amylopectin, glucose units are linked in a linear way with α(1→4) glycosidic bonds and branching takes place with α(1→6) bonds occurring every 24 to 30 glucose units. In particular, the term “starch” refers to the amylose and/or amylopectin from any plant-based material including but not limited to grains, grasses, tubers and roots and more specifically wheat, barley, corn, rye, oats, sorghum, milo, rice, sorghum, brans, cassava, millet, potato, sweet potato and tapioca.

In one embodiment, the starch is derived from raw plant material. A variety of different starch-containing raw plant materials can be employed in the starch processing methods disclosed herein. The term “raw plant material” refers to a plant material that is minimally processed or unprocessed, a grass, stalk, fruit, seed, leaf, wood, petal, fiber or any other plant part, often a feedstock or raw biomass, a plant-derived biomaterial or a plant which has undergone the transformation required to prepare it for further processing or for transport, e.g. milling, pressing, shaping, flaking.

In one embodiment, the starch is derived from cereals. In a preferred embodiment, the starch is derived from corn, i.e. is corn starch. It is well known in the art how to extract native starch from the above-mentioned plants. It does not however exclude that modified starch can be used in any of the process steps of the present invention.

The starch-containing raw plant material can, for example, be obtained as a previously treated plant product such as soy cake generated during the processing of soybeans. In some embodiments, the raw plant material is a mixture of such materials and by-products of such materials, e.g., corn fiber, corn cobs, stover, or other cellulose- and hemicellulose-containing materials, such as wood or plant residues.

Modified starch refers to chemically modified or enzymatically modified starch, or starch modified by heat treatment or by physical treatment. The term “chemically modified” includes, but is not limited to, crosslinking, modification with blocking groups to inhibit retrogradation, modification by the addition of lipophilic groups, acetylated starches, hydroxyethylated and hydroxypropylated starches, inorganically esterified starches, cationic, anionic and oxidized starches, zwitterionic starches, starches modified by enzymes and combinations thereof. Heat treatment includes for example pregelatinization.

The term “slurry” refers to a mixture of solids denser than water suspended in liquid, usually water, thus it refers to an aqueous mixture containing insoluble solids. As described, the slurry of the present invention is a starch slurry, comprising a starch and water. In a preferred embodiment, the starch present in the slurry used in the present invention is derived from corn.

Slurry parameters that the skilled person is aware of and that the skilled person can experimentally determine include concentration of solids, density and specific gravity of solids and slurry, particle size, particle distribution and particle shape.

### Alpha-Amylases

The term “alpha-amylase” (1,4-alpha-D-glucan glucanohydrolase E.C. 3.2.1.1) refers to an enzyme which acts on starch, glycogen and related polysaccharides and oligosaccharides in a random manner, by catalyzing the hydrolysis of (1→4)-alpha-D-glucosidic linkages in polysaccharides containing three or more (1→4)-alpha-linked D-glucose units. The hydrolysis of amylose or amylopectin, the polysaccharides found in starch, results in polysaccharide, oligosaccharide or glucose.

The first alpha-amylase used in the present invention is an alpha-amylase having an amino acid sequence which is at least 80% identical to the amino acid sequence according to SEQ ID NO: 1. In one embodiment, the first alpha-amylase has an amino acid sequence which is at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or at least 100% identical to the amino acid sequence according to SEQ ID NO:1. Preferably, the first alpha-amylase has an amino acid sequence which is 100% identical to the amino acid sequence according to SEQ ID NO:1.

“Sequence Identity”, “% sequence identity”, “% identity”, “% identical” or “sequence alignment” means a comparison of a first amino acid sequence to a second amino acid sequence, or a comparison of a first nucleic acid sequence to a second nucleic acid sequence and is calculated as a percentage based on the comparison. The result of this calculation can be described as “percent identical” or “percent ID.”

Generally, a sequence alignment can be used to calculate the sequence identity by one of two different approaches. In the first approach, both mismatches at a single position and gaps at a single position are counted as non-identical positions in final sequence identity calculation. In the second approach, mismatches at a single position are counted as non-identical positions in final sequence identity calculation; however, gaps at a single position are not counted (ignored) as non-identical positions in final sequence identity calculation. In other words, in the second approach gaps are ignored in final sequence identity calculation. The difference between these two approaches, i.e. counting gaps as non-identical positions vs ignoring gaps, at a single position can lead to variability in the sequence identity value between two sequences.

A sequence identity is determined by a program, which produces an alignment, and calculates identity counting both mismatches at a single position and gaps at a single position as non-identical positions in final sequence identity calculation. For example program Needle (EMBOS), which has implemented the algorithm of Needleman and Wunsch (Needleman and Wunsch, 1970, J. Mol. Biol. 48:443-453), and which calculates sequence identity by first producing an alignment between a first sequence and a second sequence, then counting the number of identical positions over the length of the alignment, then dividing the number of identical residues by the length of an alignment, then multiplying this number by 100 to generate the % sequence identity [% sequence identity=(# of Identical residues/length of alignment)×100)].

A sequence identity can be calculated from a pairwise alignment showing both sequences over the full length, so showing the first sequence and the second sequence in their full length (“Global sequence identity”). For example, program Needle (EMBOSS) produces such alignments; % sequence identity=(# of identical residues/length of alignment)×100)].

A sequence identity can be calculated from a pairwise alignment showing only a local region of the first sequence or the second sequence (“Local Identity”). For example, program Blast (NCBI) produces such alignments; % sequence identity=(# of Identical residues/length of alignment)×100)].

A sequence alignment is calculated wherein mismatches at a single position are counted as non-identical positions in final sequence identity calculation; however, gaps at a single position are not counted (ignored) as non-identical positions in final sequence identity calculation. The sequence alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used with the programs default parameter (gap open=10.0, gap extend-0.5 and matrix=EBLOSUM62). Then, a sequence identity can be calculated from the alignment showing both sequences over the full length, so showing the first sequence and the second sequence in their full length (“Global sequence identity”). For example: % sequence identity=(# of identical residues/length of alignment)×100)].

In one embodiment, the first alpha-amylase is a variant polypeptide of the alpha-amylase according to any one of SEQ ID NOs. 1, 3, 4, 5, 6 and 7.

The variant polypeptides are described by reference to an amino acid sequence which is at least n % identical to the amino acid sequence of the respective parent enzyme with “n” being an integer between 80 and 100. The variant polypeptides include enzymes that are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical when compared to the full length amino acid sequence of the parent alpha-amylase according to any one of SEQ ID Nos. 1, 3, 4, 5, 6 and 7, wherein the variant polypeptide has alpha-amylase activity.

In one embodiment, the variant polypeptide comprises at least one amino acid modification at an amino acid residue position number selected from the group consisting of: 23, 33, 181, 260, 272, 323, 349, 357, 407, and 408 or a combination thereof in the numbering of any one of SEQ ID Nos. 1, 3, 4, 5, 6 and 7.

The term “amino acid modification” means that the amino acid sequence of the variant polypeptide is modified compared to the amino acid sequence of the parent polypeptide, i.e. the polypeptide according to any one of SEQ ID Nos. 1, 3, 4, 5, 6 and 7. The term “amino acid modification” is not intended to comprise modifications to an amino acid residue itself, such as, but not limited to, phosphorylation, myristoylation, palmitoylation, isoprenylation, acetylation, alkylation, amidation, gamma-carboxylation or glycoslation. The term “amino acid modification” includes amino acid substitution, amino acid insertion and amino acid deletion. Hence, the variant polypeptide of the present invention comprises at least one amino acid substitution, amino acid insertion and/or amino acid deletion compared to the parent polypeptide, i.e. the polypeptide according to any one of SEQ ID Nos. 1, 3, 4, 5, 6 and 7. Preferably, the amino acid modification is an amino acid substitution.

“Amino acid substitutions” may be described by providing the original amino acid residue in the parent polypeptide followed by the number of the position of this amino acid residue within the amino acid sequence. For example, a substitution of amino acid residue 23 means that the amino acid of the parent at position 23 can be substituted with any of the 19 other amino acid residues and is designated as “23”. In addition, a substitution can be described by providing the original amino acid residue in the parent polypeptide. For example, the substitution of serine at residue 23 is designated as “Ser23” or “S23”. In addition, a substitution can be described by providing the original amino acid residue in the parent polypeptide followed by the number of the position of this amino acid residue within the amino acid sequence and followed by the specific substituted amino acid within the variant polypeptide. For example, the substitution of serine at position 23 with glutamate is designated as “Ser23Glu” or “S23E”. In addition, a substitution can be described by providing the number of the position of this amino acid residue within the amino acid sequence and followed by the specific substituted amino acid within the variant polypeptide. For example, the substitution at position 23 with glutamate is designated as “23Glu” or “23E”. If more than one specific amino acid substitution follows the position number, e.g. “260D/E”, the parent amino acid at the indicated position (here: position 260) can be substituted by any one of the listed substituted amino acids (here: either aspartic acid or glutamic acid). Combinations of substitutions are described by inserting commas between the amino acid residues, for example: 23E, 260E, 272E, S407E represents a combination of substitutions of four different amino acid residues when compared to a parent polypeptide. Variants having a substitution on the amino acid level are encoded by a nucleic acid sequence which differs from the parent nucleic acid sequence encoding the parent polypeptide at least in the position encoding the substituted amino acid residue.

The amino acid substitution in the variant polypeptide may be a conservative amino acid substitution. A “conservative amino acid substitution” or “substitution with a related amino acid” means replacement of one amino acid residue in an amino acid sequence with a different amino acid residue having a similar property at the same position compared to the parent amino acid sequence. Some examples of a conservative amino acid substitution include, but are not limited to, replacing a positively charged amino acid residue with a different positively charged amino acid residue; replacing a polar amino acid residue with a different polar amino acid residue; replacing a non-polar amino acid residue with a different non-polar amino acid residue, replacing a basic amino acid residue with a different basic amino acid residue, or replacing an aromatic amino acid residue with a different aromatic amino acid residue.

A list of conservative amino acid substitutions is provided in the Table below (see for example Creighton (1984) Proteins. W.H. Freeman and Company (Eds)).

| Residue | Conservative Substitution(s) |
|---|---|
| Ala | Ser |
| Arg | Lys |
| Asn | Gln, His |
| Asp | Glu |
| Gln | Asn |
| Cys | Ser |
| Glu | Asp |
| Gly | Pro |
| His | Asn, Gln |
| Ile | Leu, Val |
| Leu | Ile, Val |
| Lys | Arg, Gln |
| Met | Leu, Ile |
| Phe | Met, Leu, Tyr |
| Ser | Thr, Gly |
| Thr | Ser, Val |
| Trp | Tyr |
| Tyr | Trp, Phe |
| Val | Ile, Leu |

In one embodiment, the variant polypeptide comprises at least one amino acid substitution selected from the group consisting of: 23E, 33E, 181E, 260D/E, 272D/E, 323E, 349P, 357E, 407E and 408E or a combination thereof in the numbering of any one of SEQ ID Nos. 1, 3, 4, 5, 6 and 7.

In a preferred embodiment, the variant polypeptide comprises the amino acid modifications of:
a) 260D, or
b) 357E, or
c) 407E, or
d) 408E, or
e) 23E, 33E, 181E, 260E, 272D, 323E, 349P, 357E, and 407E, or
f) 23E, 260E, 272E, and 407E
   in the numbering of any one of SEQ ID Nos. 1, 3, 4, 5, 6 and 7.

In one embodiment, the variant polypeptide is a variant of the alpha-amylase according to SEQ ID NO: 3 and comprises at least one amino acid substitution selected from the group consisting of: G23E, S33E, D181E, N260D/E, Q272D/E, N323E, S349P, N357E, S407E and S408E or a combination thereof in the numbering of SEQ ID NO: 3.

In a preferred embodiment, the variant polypeptide of the alpha-amylase according to SEQ ID NO: 3 comprises the amino acid modifications of:
a) N260D, or
b) N357E
c) S408E, or
d) G23E, S33E, D181E, N260E, Q272D, N323E, S349P, N357E, and S407E, or
e) G23E, N260E, Q272E, and S407E in the numbering of SEQ ID NO: 3.

In one embodiment, the variant polypeptide is a variant of the alpha-amylase according to SEQ ID NO: 4 and comprises at least one amino acid substitution selected from the group consisting of: M23E, Q33E, Q181E, N260D/E, Q272D/E, N323E, N349P, N357E, S407E and S408E or a combination thereof in the numbering of SEQ ID NO: 4.

In a preferred embodiment, the variant polypeptide of the alpha-amylase according to SEQ ID NO: 4 comprises the amino acid modifications of:
a) N260D, or
b) N357E
c) S408E, or
d) M23E, Q33E, Q181E, N260E, Q272D, N323E, N349P, N357E, and S407E, or
e) M23E, N260E, Q272E, and S407E in the numbering of SEQ ID NO: 4.

In one embodiment, the variant polypeptide is a variant of the alpha-amylase according to SEQ ID NO: 5 and comprises at least one amino acid substitution selected from the group consisting of: S23E, Q33E, N181E, N260D/E, G272D/E, N323E, N349P, N357E, S407E and S408E or a combination thereof in the numbering of SEQ ID NO: 5.

In a preferred embodiment, the variant polypeptide of the alpha-amylase according to SEQ ID NO: 5 comprises the amino acid modifications of:
a) N260D, or
b) N357E, or
c) S408E, or
d) S23E, Q33E, N181E, N260E, G272D, N323E, N349P, N357E, and S407E, or
e) S23E, N260E, G272E, and S407E in the numbering of SEQ ID NO: 5.

In one embodiment, the variant polypeptide is a variant of the alpha-amylase according to SEQ ID NO: 6 and comprises at least one amino acid substitution selected from the group consisting of: N260D, N357E, and S408E or a combination thereof in the numbering of SEQ ID NO: 6.

In a preferred embodiment, the variant polypeptide of the alpha-amylase according to SEQ ID NO: 6 comprises the amino acid modifications of:
a) N260D, or
b) N357E, or
c) S408E, in the numbering of SEQ ID NO: 6.

In one embodiment, the variant polypeptide is a variant of the alpha-amylase according to SEQ ID NO: 7 and comprises at least one amino acid substitution selected from the group consisting of: G23E, S33E, N181E, N260D/E, Q272D/E, N323E, N349P, N357E, S407E and S408E or a combination thereof in the numbering of SEQ ID NO: 7.

In a preferred embodiment, the variant polypeptide of the alpha-amylase according to SEQ ID NO: 7 comprises the amino acid modifications of:
a) N260D, or
b) N357E, or
c) S408E, or
d) G23E, S33E, N181E, N260E, Q272D, N323E, N349P, N357E, and S407E, or
e) G23E, N260E, Q272E, and S407E in the numbering of SEQ ID NO: 7.

In a preferred embodiment, the variant polypeptide of the alpha-amylase according to SEQ ID NO: 1 comprises the amino acid modifications of:
a) N260D, or
b) N357E, or
c) S407E
d) S408E, or
e) G23E, S33E, N181E, N260E, Q272D, N323E, N349P, N357E, and S407E, or
f) G23E, N260E, Q272E, and S407E in the numbering of SEQ ID NO: 1.

In one embodiment, the first alpha-amylase used in the present invention is present in an amount of from about 0.001% to about 0.06%, about 0.005% to about 0.06%, about 0.01% to about 0.06%, about 0.015% to about 0.06%, about 0.02% to about 0.06%, about 0.025% to about 0.06%, about 0.03% to about 0.06%, about 0.035% to about 0.06%, about 0.04% to about 0.06%, about 0.045% to about 0.06%, about 0.001% to about 0.05%, about 0.005% to about 0.05%, about 0.01% to about 0.05%, about 0.015% to about 0.05%, about 0.02% to about 0.05%, about 0.025% to about 0.05%, about 0.03% to about 0.05%, about 0.035% to about 0.05%, about 0.04% to about 0.05%, about 0.045% to about 0.05%, about 0.001% to about 0.045%, about 0.005% to about 0.045%, about 0.01% to about 0.045%, about 0.015% to about 0.045%, about 0.02% to about 0.045%, about 0.025% to about 0.045%, about 0.03% to about 0.045%, about 0.035% to about 0.045%, about 0.04% to about 0.045%, about 0.001% to about 0.04%, about 0.005% to about 0.04%, about 0.01% to about 0.04%, about 0.015% to about 0.04%, about 0.02% to about 0.04%, about 0.025% to about 0.04%, about 0.03% to about 0.04%, about 0.035% to about 0.04%, about 0.001% to about 0.035, about 0.005% to about 0.035%, about 0.01% to about 0.035%, about 0.015% to about 0.035%, about 0.02% to about 0.035%, about 0.025% to about 0.035%, about 0.03% to about 0.035%, about 0.001% to about 0.03, about 0.005% to about 0.03%, about 0.01% to about 0.03%, about 0.015% to about 0.03%, about 0.02% to about 0.03%, about 0.025% to about 0.03%, about 0.001% to about 0.025%, about 0.005% to about 0.025%, about 0.01% to about 0.025%, about 0.015% to about 0.025%, about 0.02% to about 0.025%, about 0.001% to about 0.02%, about 0.005% to about 0.02%, about 0.01% to about 0.02%, about 0.015% to about 0.02%, about 0.001% to about 0.015, about 0.005% to about 0.015%, about 0.01% to about 0.015%, about 0.001% to about 0.01%, about 0.005% to about 0.01%, about 0.001% to about 0.005% weight of enzyme by weight of raw plant material. In a preferred embodiment, the first alpha-amylase is present in an amount from about 0.005% to about 0.01% weight of enzyme by weight of raw plant material.

In another embodiment, the first alpha-amylase is present in an amount of about 0.001%, about 0.002%, about 0.003% about 0.004%, about 0.005%, about 0.006%, about 0.007%, about 0.008%, about 0.009%, about 0.01%, 0.011%, about 0.012%, about 0.013% about 0.014%, about 0.015%, about 0.016%, about 0.017%, about 0.018%, about 0.019%, about 0.02%, 0.021%, about 0.022%, about 0.023% about 0.024%, about 0.025%, about 0.026%, about 0.027%, about 0.028%, about 0.029%, about 0.03%, 0.031%, about 0.032%, about 0.033% about 0.034%, about 0.035%, about 0.036%, about 0.037%, about 0.038%, about 0.039%, about 0.04%, 0.041%, about 0.042%, about 0.043% about 0.044%, about 0.045%, about 0.046%, about 0.047%, about 0.048%, about 0.049%, about 0.05%, about 0.051%, about 0.052%, about 0.053%, about 0.054%, about 0.055%, about 0.056%, about 0.057%, about 0.058%, about 0.059% or about 0.06% weight of enzyme by weight of raw plant material. In a preferred embodiment, the first alpha-amylase is present in an amount of about 0.02% by weight of raw plant material.

The second alpha-amylase of the invention is an alpha-amylase which is different from the first alpha-amylase. The second alpha amylase can be derived from any animal, plant or microbial source or it can be a synthetic or recombinant alpha-amylase. In one embodiment, the second alpha-amylase is a thermostable alpha-amylase.

The term “thermostable” refers to an enzyme that is not subject to destruction or alteration by heat. In one embodiment, the thermostable alpha-amylase retains at least 20%, at least 30%, at least 40%, at least 50%, at least 60%, at least 70%, at least 80%, at least 90% or 100% of its relative activity at temperatures of at or above 50° C., at or above 60° C., at or above 70° C., at or above 80° C., at or above 85° C., at or above 90° C., at or above 95° C. or at or above 100%.

In one embodiment, the second alpha-amylase is an alpha-amylase derived from a microorganism, preferably from a Gram-positive bacterium, most preferably from a Gram-positive bacterium from the genus Bacillus. In one embodiment, the second alpha amylase is derived from Geobacillus stearothermophilus, Bacillus licheniformis, Bacillus subtilis, Bacillus amyloliquefaciens, Bacillus acidocaldarius or Bacillus circulans. The skilled person is aware of methods to isolate alpha-amylases from microorganisms. Alpha-amylases may be produced in, i.e. expressed in and isolated from, recombinant prokaryotic or eukaryotic cells by any method known to the skilled person.

In a preferred embodiment, the second alpha-amylase is an alpha-amylase from Geobacillus stearothermophilus or a variant thereof. In one embodiment, the second alpha-amylase is an alpha-amylase comprising an amino acid sequence which is at least 80% identical to the amino acid sequence according to SEQ ID NO:2. In one embodiment, the second alpha-amylase is at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence according to SEQ ID NO:2. In a preferred embodiment, the second alpha-amylase is an alpha-amylase comprising an amino acid sequence which is at least 80% identical to the amino acid sequence according to SEQ ID NO: 2.

In one embodiment, the second alpha-amylase comprises an amino acid sequence which is at least 80% identical, is at least 81%, at least 82%, at least 83%, at least 84%, at least 85%, at least 86%, at least 87%, at least 88%, at least 89%, at least 90%, at least 91%, at least 92%, at least 93%, at least 94% or at least 95% identical to the amino acid sequence according to SEQ ID NO:2 and comprises at least amino acids 142 to 175 of the amino acid sequence according to SEQ ID NO:2.

In another embodiment, the second alpha-amylase is derived from a microorganism, wherein the microorganism is a fungus. In a preferred embodiment, the second alpha-amylase is derived from a fungus of the genus Aspergillus.

The second alpha-amylase may be a commercial alpha-amylase. In one embodiment, the second alpha-amylase is SUKAMY HI available from Shandong Sukahan Bio-Technology, HTAA40L or HTAA180L available from Sunson®, MEGA TAL-18 available from PeliBioTech or HTA CON 2 available from HYX. In a preferred embodiment, the second alpha-amylase is SUKAMY HI available from Shandong Sukahan Bio-Technology. Other suitable second alpha-amylases are present in the products Avantec® Amp, BAN™ and Liquozyme® SCDC, available from Novozymes, AMYL-LP, AMYL-LP Strong, AMYL-XT, XT-SR and SZM XT-20+ available from CTE Global Inc, SPEZYME® AA and SPEZYME® FRED available from Genencor®.

In one embodiment, the first alpha-amylase is a variant polypeptide of the alpha-amylase according to SEQ ID No. 3 which comprises the amino acid substitutions 23E, 33E, 181E, 260E, 272D, 323E, 357E, 349P and 407E, the numbering referring to SEQ ID NO: 3, and the second alpha-amylase comprises an amino acid sequence which is at least 80% identical to the amino acid sequence according to SEQ ID NO:2.

In one embodiment, the first alpha-amylase is a variant polypeptide of the alpha-amylase according to SEQ ID No. 3 which comprises the amino acid substitutions 23E, 260E, 272E and 407E, the numbering referring to SEQ ID NO: 3, and the second alpha-amylase comprises an amino acid sequence which is at least 80% identical to the amino acid sequence according to SEQ ID NO:2.

In one embodiment, the first alpha-amylase is a variant polypeptide of the alpha-amylase according to SEQ ID No. 4 which comprises the amino acid substitution 357E, the numbering referring to SEQ ID NO: 4, and the second alpha-amylase comprises an amino acid sequence which is at least 80% identical to the amino acid sequence according to SEQ ID NO: 2.

In one embodiment, the first alpha-amylase is a variant polypeptide of the alpha-amylase according to SEQ ID No. 4 which comprises the amino acid substitution 408E, the numbering referring to SEQ ID NO: 4, and the second alpha-amylase comprises an amino acid sequence which is at least 80% identical to the amino acid sequence according to SEQ ID NO: 2.

In one embodiment, the first alpha-amylase is a variant polypeptide of the alpha-amylase according to SEQ ID No. 5 which comprises the amino acid substitution 357E, the numbering referring to SEQ ID NO: 5, and the second alpha-amylase comprises an amino acid sequence which is at least 80% identical to the amino acid sequence according to SEQ ID NO: 2.

In one embodiment, the first alpha-amylase is a variant polypeptide of the alpha-amylase according to SEQ ID No. 5 which comprises the amino acid substitution 408E, the numbering referring to SEQ ID NO: 5, and the second alpha-amylase comprises an amino acid sequence which is at least 80% identical to the amino acid sequence according to SEQ ID NO: 2.

In one embodiment, the first alpha-amylase is a variant polypeptide of the alpha-amylase according to SEQ ID No. 6 which comprises the amino acid substitution 357E, the numbering referring to SEQ ID NO: 6, and the second alpha-amylase comprises an amino acid sequence which is at least 80% identical to the amino acid sequence according to SEQ ID NO: 2.

In one embodiment, the first alpha-amylase is a variant polypeptide of the alpha-amylase according to SEQ ID No. 6 which comprises the amino acid substitution 408E, the numbering referring to SEQ ID NO: 6, and the second alpha-amylase comprises an amino acid sequence which is at least 80% identical to the amino acid sequence according to SEQ ID NO: 2.

In one embodiment, the first alpha-amylase is a variant polypeptide of the alpha-amylase according to SEQ ID No. 1 which comprises the amino acid substitution 407E, the numbering referring to SEQ ID NO: 1, and the second alpha-amylase comprises an amino acid sequence which is at least 80% identical to the amino acid sequence according to SEQ ID NO: 2.

The “inclusion rate” of an enzyme refers to the amount of said enzyme compared to the total enzyme dose and is expressed in percent (%) of the total enzyme dose. In one embodiment, the second alpha-amylase is present at an inclusion rate of 1% to 50%, 5% to 50%, 10% to 50%, 15% to 50%, 20% to 50%, 25% to 50%, 30% to 50%, 35% to 50%, 40% to 50%, 45% to 50%, 1% to 45%, 5% to 45%, 10% to 45%, 15% to 45%, 20% to 45%, 25% to 45%, 30% to 45%, 35% to 45%, 40% to 45%, 1% to 40%, 5% to 40%, 10% to 40%, 15% to 40%, 20% to 40%, 25% to 40%, 30% to 40%, 35% to 40%, 1% to 35%, 5% to 35%, 10% to 35%, 15% to 35%, 20% to 35%, 25% to 35%, 30% to 35%, 1% to 30%, 5% to 30%, 10% to 30%, 15% to 30%, 20% to 30%, 25% to 30%, 1% to 25%, 5% to 25%, 10% to 25%, 15% to 25%, 20% to 25%, 1% to 20%, 5% to 20%, 10% to 20%, 1% to 15%, 5% to 15%, 10% to 15%, 1% to 10%, 5% to 10%, or 1% to 5%. In a preferred embodiment, the second alpha-amylase is present at an inclusion rate of 15% to 20%.

In another embodiment, the second alpha-amylase is present is present at an inclusion rate of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In a preferred embodiment, the second alpha-amylase is present at an inclusion rate of about 20%.

In one embodiment, the weight ratio of the first alpha-amylase to the second alpha-amylase is between about 20:1 to about 1:10. The “weight ratio” refers to the mass of the first alpha-amylase divided by the mass of the second alpha amylase. In one embodiment, the weight ratio of the first alpha-amylase to the second alpha-amylase is between about 20:1 to about 1:5, about 20:1 to about 1:1, about 20:1 to about 5:1, about 20:1 to about 10:1, about 20:1 to about 15:1, about 15:1 to about 1:10, about 15:1 to about 1:5, about 15:1 to about 1:1, about 15:1 to about 5:1, about 15:1 to about 10:1, about 10:1 to about 1:10, about 10:1 to about 1:5, about 10:1 to about 1:1, about 10:1 to about 5:1, about 5:1 to about 1:10, about 5:1 to about 1:5, about 5:1 to about 1:1, about 1:1 to about 1:10, about 1:1 to about 1:5. In a preferred embodiment, the weight ratio of the first alpha-amylase to the second alpha-amylase is between about 5:1 to 1:5.

In another embodiment, the weight ratio of the first alpha-amylase to the second alpha-amylase is about 20:1, about 15:1, about 10:1, about 5:1, about 1:1, about 1:5 or about 1:10. In a preferred embodiment, the weight ratio of the first alpha-amylase to the second alpha-amylase is about 5:1.

In one embodiment, the total enzyme dose of the first and the second alpha-amylase is between about 0.002% to about 0.05% total weight of enzyme by weight of raw plant material. The total weight of enzyme by weight of raw plant material in % is calculated as follows: total weight enzyme÷total weight raw plant material×100. In one embodiment, the total enzyme dose of the first and the second alpha-amylase is between about 0.005% to about 0.06%, 0.007% to about 0.06%, 0.01% to about 0.06%, 0.012% to about 0.06%, 0.015% to about 0.06%, 0.017% to about 0.06%, 0.02% to about 0.06%, 0.022% to about 0.06%, 0.025% to about 0.06%, 0.027% to about 0.06%, 0.03% to about 0.06%, 0.032% to about 0.06%, 0.035% to about 0.06%, 0.04% to about 0.06%, 0.042% to about 0.06%, 0.045% to about 0.06%, 0.047% to about 0.06%, 0.005% to about 0.05%, 0.007% to about 0.05%, 0.01% to about 0.05%, 0.012% to about 0.05%, 0.015% to about 0.05%, 0.017% to about 0.05%, 0.02% to about 0.05%, 0.022% to about 0.05%, 0.025% to about 0.05%, 0.027% to about 0.05%, 0.03% to about 0.05%, 0.032% to about 0.05%, 0.035% to about 0.05%, 0.04% to about 0.05%, 0.042% to about 0.05%, 0.045% to about 0.05%, 0.047% to about 0.05%, about 0.002% to about 0.045%, 0.005% to about 0.045%, 0.007% to about 0.045%, 0.01% to about 0.045%, 0.012% to about 0.045%, 0.015% to about 0.045%, 0.017% to about 0.045%, 0.02% to about 0.045%, 0.022% to about 0.045%, 0.025% to about 0.045%, 0.027% to about 0.045%, 0.03% to about 0.045%, 0.032% to about 0.045%, 0.035% to about 0.045%, 0.04% to about 0.045%, 0.042% to about 0.045%, about 0.002% to about 0.04%, 0.005% to about 0.04%, 0.007% to about 0.04%, 0.01% to about 0.04%, 0.012% to about 0.04%, 0.015% to about 0.04%, 0.017% to about 0.04%, 0.02% to about 0.04%, 0.022% to about 0.04%, 0.025% to about 0.04%, 0.027% to about 0.04%, 0.03% to about 0.04%, 0.032% to about 0.04%, 0.035% to about 0.04%, about 0.002% to about 0.035%, 0.005% to about 0.035%, 0.007% to about 0.035%, 0.01% to about 0.035%, 0.012% to about 0.035%, 0.015% to about 0.035%, 0.017% to about 0.035%, 0.02% to about 0.035%, 0.022% to about 0.035%, 0.025% to about 0.035%, 0.027% to about 0.035%, 0.03% to about 0.035%, 0.032% to about 0.035%, about 0.002% to about 0.03%, 0.005% to about 0.03%, 0.007% to about 0.03%, 0.01% to about 0.03%, 0.012% to about 0.03%, 0.015% to about 0.03%, 0.017% to about 0.03%, 0.02% to about 0.03%, 0.022% to about 0.03%, 0.025% to about 0.03%, 0.027% to about 0.03%, about 0.002% to about 0.025%, 0.005% to about 0.025%, 0.007% to about 0.025%, 0.01% to about 0.025%, 0.012% to about 0.025%, 0.015% to about 0.025%, 0.017% to about 0.025%, 0.02% to about 0.025%, 0.022% to about 0.025%, about 0.002% to about 0.02%, 0.005% to about 0.02%, 0.007% to about 0.02%, 0.01% to about 0.02%, 0.012% to about 0.02%, 0.015% to about 0.02%, 0.017% to about 0.02%, about 0.002% to about 0.015%, 0.005% to about 0.015%, 0.007% to about 0.015%, 0.01% to about 0.015%, 0.012% to about 0.015%, about 0.002% to about 0.01%, 0.005% to about 0.01%, 0.007% to about 0.01%, or 0.002% to about 0.005% total weight of enzyme by weight of raw plant material. In a preferred embodiment, the total enzyme dose of the first and the second alpha-amylase is between about 0.005% to about 0.06% total weight of enzyme by weight of raw plant material.

In another embodiment, the total enzyme dose of the first and the second alpha-amylase is about 0.002%, about 0.003% about 0.004%, about 0.005%, about 0.006%, about 0.007%, about 0.008%, about 0.009%, about 0.01%, 0.011%, about 0.012%, about 0.013% about 0.014%, about 0.015%, about 0.016%, about 0.017%, about 0.018%, about 0.019%, about 0.02%, 0.021%, about 0.022%, about 0.023% about 0.024%, about 0.025%, about 0.026%, about 0.027%, about 0.028%, about 0.029%, about 0.03%, 0.031%, about 0.032%, about 0.033% about 0.034%, about 0.035%, about 0.036%, about 0.037%, about 0.038%, about 0.039%, about 0.04%, 0.041%, about 0.042%, about 0.043% about 0.044%, about 0.045%, about 0.046%, about 0.047%, about 0.048%, about 0.049%, or about 0.05%, %, about 0.051%, about 0.052%, about 0.053%, about 0.054%, about 0.055%, about 0.056%, about 0.057%, about 0.058%, about 0.059% or about 0.06% total weight of enzyme by weight of raw plant material. In a preferred embodiment, the total enzyme dose of the first and the second alpha-amylase is about 0.02% total weight of enzyme by weight of raw plant material.

The term “adding” refers to the placing of the respective enzyme(s) in sufficiently close proximity to the respective substrate to enable the enzyme(s) to convert the substrate to the end-product. In the context of the present invention “adding” refers to the placing of the first and the second alpha-amylase in sufficiently close proximity to starch to enable the alpha-amylases to convert the starch to shorter fragments.

In one embodiment, the first alpha-amylase and the second alpha-amylase are added to the slurry simultaneously. “Simultaneously” means that the first alpha-amylase and the second alpha-amylase are added to the slurry at the same time. The enzymes may be added from separate containers simultaneously. Alternatively, the first alpha-amylase and the second alpha-amylase may be pre-mixed or blended before being added simultaneously.

In another embodiment, the first alpha-amylase and the second alpha-amylase are added separately. The first alpha-amylase and the second alpha-amylase may be added one after the other. The first alpha-amylase and the second alpha-amylase may be added at different stages of the process. The first alpha-amylase and the second alpha-amylase may be added up to 90 minutes apart. In one embodiment, the first alpha-amylase and the second alpha-amylase are added 5 minutes apart, between 5 and 10 minutes apart, between 10 and 20 minutes apart, between 20 and 30 minutes apart, between 30 and 40 minutes apart, between 40 and 50 minutes apart, between 50 and 60 minutes apart, between 60 and 70 minutes apart, between 70 and 80 minutes apart or between 80 and 90 minutes apart.

In one embodiment, the first alpha-amylase and the second alpha-amylase are added separately and simultaneously, i.e. the first alpha-amylase is added as a first solution and the second alpha-amylase is added to the slurry at the same time, but as part of a second solution.

In one embodiment, the total enzyme dose of the first and the second alpha-amylase is about 0.02% total weight of enzyme by weight of raw plant material and the second alpha-amylase is present at an inclusion rate of about 5%; the total enzyme dose of the first and the second alpha-amylase is about 0.02% total weight of enzyme by weight of raw plant material and the second alpha-amylase is present at an inclusion rate of about 10%; the total enzyme dose of the first and the second alpha-amylase is about 0.02% total weight of enzyme by weight of raw plant material and the second alpha-amylase is present at an inclusion rate of about 15%; the total enzyme dose of the first and the second alpha-amylase is about 0.02% total weight of enzyme by weight of raw plant material and the second alpha-amylase is present at an inclusion rate of about 20%; the total enzyme dose of the first and the second alpha-amylase is about 0.03% total weight of enzyme by weight of raw plant material and the second alpha-amylase is present at an inclusion rate of about 5%; the total enzyme dose of the first and the second alpha-amylase is about 0.03% total weight of enzyme by weight of raw plant material and the second alpha-amylase is present at an inclusion rate of about 10%; the total enzyme dose of the first and the second alpha-amylase is about 0.03% total weight of enzyme by weight of raw plant material and the second alpha-amylase is present at an inclusion rate of about 15%; or the total enzyme dose of the first and the second alpha-amylase is about 0.03% total weight of enzyme by weight of raw plant material and the second alpha-amylase is present at an inclusion rate of about 20%; the total enzyme dose of the first and the second alpha-amylase is about 0.06% total weight of enzyme by weight of raw plant material and the second alpha-amylase is present at an inclusion rate of about 5%; the total enzyme dose of the first and the second alpha-amylase is about 0.06% total weight of enzyme by weight of raw plant material and the second alpha-amylase is present at an inclusion rate of about 10%; the total enzyme dose of the first and the second alpha-amylase is about 0.06% total weight of enzyme by weight of raw plant material and the second alpha-amylase is present at an inclusion rate of about 15%; or the total enzyme dose of the first and the second alpha-amylase is about 0.06% total weight of enzyme by weight of raw plant material and the second alpha-amylase is present at an inclusion rate of about 20%.

In one embodiment, the first alpha-amylase is present in an amount of about 0.0035% total weight of enzyme by weight of raw plant material and the second alpha-amylase is present in an amount of about 0.002% total weight of enzyme by weight of raw plant material.

In one embodiment, the first alpha-amylase is present in an amount of about 0.0069% total weight of enzyme by weight of raw plant material and the second alpha-amylase is present in an amount of about 0.0012% total weight of enzyme by weight of raw plant material.

In one embodiment, the first alpha-amylase is present in an amount of about 0.0019% total weight of enzyme by weight of raw plant material and the second alpha-amylase is present in an amount of about 0.0037% total weight of enzyme by weight of raw plant material.

In one embodiment, the first alpha-amylase is present in an amount of about 0.0057% total weight of enzyme by weight of raw plant material and the second alpha-amylase is present in an amount of about 0.0044% total weight of enzyme by weight of raw plant material.

In one embodiment, the first alpha-amylase is present in an amount of about 0.01% total weight of enzyme by weight of raw plant material and the second alpha-amylase is present in an amount of about 0.0054% total weight of enzyme by weight of raw plant material.

In one embodiment, the first alpha-amylase is present in an amount of about 0.0065% total weight of enzyme by weight of raw plant material and the second alpha-amylase is present in an amount of about 0.0083% total weight of enzyme by weight of raw plant material.

In one embodiment, the first alpha-amylase is present in an amount of about 0.003% total weight of enzyme by weight of raw plant material and the second alpha-amylase is present in an amount of about 0.01% total weight of enzyme by weight of raw plant material.

In one embodiment, the first alpha-amylase is present in an amount of about 0.01% total weight of enzyme by weight of raw plant material and the second alpha-amylase is present in an amount of about 0.01% total weight of enzyme by weight of raw plant material.

In one embodiment, the first alpha-amylase is present in an amount of about 0.009% total weight of enzyme by weight of raw plant material and the second alpha-amylase is present in an amount of about 0.001% total weight of enzyme by weight of raw plant material.

In one embodiment, the first alpha-amylase is present in an amount of about 0.009% total weight of enzyme by weight of raw plant material and the second alpha-amylase is present in an amount of about 0.002% total weight of enzyme by weight of raw plant material.

In one embodiment, the first alpha-amylase is present in an amount of about 0.007% total weight of enzyme by weight of raw plant material and the second alpha-amylase is present in an amount of about 0.003% total weight of enzyme by weight of raw plant material.

In one embodiment, the first alpha-amylase is present in an amount of about 0.0185% total weight of enzyme by weight of raw plant material and the second alpha-amylase is present in an amount of about 0.0015% total weight of enzyme by weight of raw plant material.

In one embodiment, the first alpha-amylase is present in an amount of about 0.017% total weight of enzyme by weight of raw plant material and the second alpha-amylase is present in an amount of about 0.003% total weight of enzyme by weight of raw plant material.

In one embodiment, the first alpha-amylase is present in an amount of about 0.0155% total weight of enzyme by weight of raw plant material and the second alpha-amylase is present in an amount of about 0.0045% total weight of enzyme by weight of raw plant material.

In one embodiment, the first alpha-amylase is present in an amount of about 0.014% total weight of enzyme by weight of raw plant material and the second alpha-amylase is present in an amount of about 0.006% total weight of enzyme by weight of raw plant material.

In one embodiment, the first alpha-amylase is present in an amount of about 0.055% total weight of enzyme by weight of raw plant material and the second alpha-amylase is present in an amount of about 0.0015% total weight of enzyme by weight of raw plant material.

In one embodiment, the first alpha-amylase is present in an amount of about 0.05% total weight of enzyme by weight of raw plant material and the second alpha-amylase is present in an amount of about 0.003% total weight of enzyme by weight of raw plant material.

In one embodiment, the first alpha-amylase is present in an amount of about 0.045% total weight of enzyme by weight of raw plant material and the second alpha-amylase is present in an amount of about 0.0045% total weight of enzyme by weight of raw plant material.

In one embodiment, the first alpha-amylase is present in an amount of about 0.04% total weight of enzyme by weight of raw plant material and the second alpha-amylase is present in an amount of about 0.006% total weight of enzyme by weight of raw plant material.

In one embodiment, the first alpha-amylase is present in an amount of about 0.03% total weight of enzyme by weight of raw plant material and the second alpha-amylase is present in an amount of about 0.0015% total weight of enzyme by weight of raw plant material.

In one embodiment, the first alpha-amylase is present in an amount of about 0.027% total weight of enzyme by weight of raw plant material and the second alpha-amylase is present in an amount of about 0.003% total weight of enzyme by weight of raw plant material.

In one embodiment, the first alpha-amylase is present in an amount of about 0.024% total weight of enzyme by weight of raw plant material and the second alpha-amylase is present in an amount of about 0.0045% total weight of enzyme by weight of raw plant material.

In one embodiment, the first alpha-amylase is present in an amount of about 0.02% total weight of enzyme by weight of raw plant material and the second alpha-amylase is present in an amount of about 0.006% total weight of enzyme by weight of raw plant material.

In one embodiment, the first alpha-amylase is present in an amount of about 0.0285% total weight of enzyme by weight of raw plant material and the second alpha-amylase is present in an amount of about 0.0015% total weight of enzyme by weight of raw plant material.

In one embodiment, the first alpha-amylase is present in an amount of about 0.027% total weight of enzyme by weight of raw plant material and the second alpha-amylase is present in an amount of about 0.003% total weight of enzyme by weight of raw plant material.

In one embodiment, the first alpha-amylase is present in an amount of about 0.0255% total weight of enzyme by weight of raw plant material and the second alpha-amylase is present in an amount of about 0.0045% total weight of enzyme by weight of raw plant material.

In one embodiment, the first alpha-amylase is present in an amount of about 0.025% total weight of enzyme by weight of raw plant material and the second alpha-amylase is present in an amount of about 0.006% total weight of enzyme by weight of raw plant material.

In one embodiment, the first alpha-amylase is present in an amount from about 0.019% total weight of enzyme by weight of raw plant material and the second alpha-amylase is present at an inclusion rate of about 5% and the total enzyme dose is 0.02%; the first alpha-amylase is present in an amount from about 0.018% total weight of enzyme by weight of raw plant material and the second alpha-amylase is present at about 10% inclusion rate and the total enzyme dose is 0.02%; the first alpha-amylase is present in an amount from about 0.017% total weight of enzyme by weight of raw plant material and the second alpha-amylase is present at about 15% inclusion rate and the total enzyme dose is 0.02%; the first alpha-amylase is present in an amount from about 0.016% total weight of enzyme by weight of raw plant material and the second alpha-amylase is present at about 20% inclusion rate and the total enzyme dose is 0.02%; the first alpha-amylase is present in an amount from about 0.0285% total weight of enzyme by weight of raw plant material and the second alpha-amylase is present at about 5% inclusion rate and the total enzyme dose is 0.03%; the first alpha-amylase is present in an amount from about 0.027% total weight of enzyme by weight of raw plant material and the second alpha-amylase is present at about 10% inclusion rate and the total enzyme dose is 0.03%; the first alpha-amylase is present in an amount from about 0.0255% total weight of enzyme by weight of raw plant material and the second alpha-amylase is present at about 15% inclusion rate and the total enzyme dose is 0.03%; or the first alpha-amylase is present in an amount from about 0.024% total weight of enzyme by weight of raw plant material and the second alpha-amylase is present at about 20% inclusion rate and the total enzyme dose is 0.03%.

In one embodiment, the first alpha-amylase is present in an amount from about 0.0185% total weight of enzyme by weight of raw plant material and the second alpha-amylase is present at an inclusion rate of about 5% and the total enzyme dose is 0.02%; the first alpha-amylase is present in an amount from about 0.017% total weight of enzyme by weight of raw plant material and the second alpha-amylase is present at about 10% inclusion rate and the total enzyme dose is 0.02%; the first alpha-amylase is present in an amount from about 0.0155% total weight of enzyme by weight of raw plant material and the second alpha-amylase is present at about 15% inclusion rate and the total enzyme dose is 0.02%; or the first alpha-amylase is present in an amount from about 0.014% total weight of enzyme by weight of raw plant material and the second alpha-amylase is present at about 20% inclusion rate and the total enzyme dose is 0.02%.

In one embodiment, the first alpha-amylase is present in an amount from about 0.055% total weight of enzyme by weight of raw plant material and the second alpha-amylase is present at about 5% inclusion rate and the total enzyme dose is 0.0565%; the first alpha-amylase is present in an amount from about 0.05% total weight of enzyme by weight of raw plant material and the second alpha-amylase is present at about 10% inclusion rate and the total enzyme dose is 0.053%; the first alpha-amylase is present in an amount from about 0.045% total weight of enzyme by weight of raw plant material and the second alpha-amylase is present at about 15% inclusion rate and the total enzyme dose is 0.0495%; or the first alpha-amylase is present in an amount from about 0.04% total weight of enzyme by weight of raw plant material and the second alpha-amylase is present at about 20% inclusion rate and the total enzyme dose is 0.046%.

In one embodiment, the first alpha-amylase is present in an amount from about 0.03% total weight of enzyme by weight of raw plant material and the second alpha-amylase is present at about 5% inclusion rate and the total enzyme dose is 0.0315%; the first alpha-amylase is present in an amount from about 0.027% total weight of enzyme by weight of raw plant material and the second alpha-amylase is present at about 10% inclusion rate and the total enzyme dose is 0.03%; the first alpha-amylase is present in an amount from about 0.024% total weight of enzyme by weight of raw plant material and the second alpha-amylase is present at about 15% inclusion rate and the total enzyme dose is 0.0285%; or the first alpha-amylase is present in an amount from about 0.02% total weight of enzyme by weight of raw plant material and the second alpha-amylase is present at about 20% inclusion rate and the total enzyme dose is 0.026%.

In one embodiment, the first alpha-amylase is present in an amount from about 0.0285% total weight of enzyme by weight of raw plant material and the second alpha-amylase is present at about 5% inclusion rate and the total enzyme dose is 0.03%; the first alpha-amylase is present in an amount from about 0.027% total weight of enzyme by weight of raw plant material and the second alpha-amylase is present at about 10% inclusion rate and the total enzyme dose is 0.03%; the first alpha-amylase is present in an amount from about 0.0255% total weight of enzyme by weight of raw plant material and the second alpha-amylase is present at about 15% inclusion rate and the total enzyme dose is 0.03%; or the first alpha-amylase is present in an amount from about 0.025% total weight of enzyme by weight of raw plant material and the second alpha-amylase is present at about 20% inclusion rate and the total enzyme dose is 0.03%.

### Composition Comprising the Second Alpha-Amylase and a Protease

In one embodiment, step (c) of the method of starch processing comprises adding a composition comprising the second alpha-amylase and a first protease.

The term “composition” refers to a mixture of two or more ingredients that may be produced by mechanical or chemical means.

The second alpha-amylase in the composition comprising the second alpha-amylase and a protease is defined as in the preceding section.

The term “protease” refers to an enzyme having proteolytic activity (also called peptidase). Proteases are members of class EC 3.4. Proteases include aminopeptidases (EC 3.4.11), dipeptidases (EC 3.4.13), dipeptidyl-peptidases and tripeptidyl-peptidases (EC 3.4.14), peptidyl-dipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endopeptidases of unknown catalytic mechanism (EC 3.4.99). The first protease can be derived from any animal, plant or microbial source or it can be a synthetic or recombinant protease.

In one embodiment, the first protease is a thermostable protease. In one embodiment, the first protease retains at least 20%, at least 30%, at least 40%, at least 50%, at least 60%, at least 70%, at least 80%, at least 90% or a 100% of its relative activity at temperatures of at or above 50° C., at or above 60° C., at or above 70° C., at or above 80° C., at or above 85° C., at or above 90° C., at or above 95° C. or at or above 100° C.

In one embodiment, the first protease is a protease derived from a microorganism, preferably a Gram-positive bacterium, most preferably a Gram-positive bacterium from the genus Bacillus. The skilled person is aware of methods to isolate a protease from microorganisms. The protease may be produced in, i.e. expressed in and isolated from, recombinant prokaryotic or eukaryotic cells by any method known to the skilled person.

In one embodiment, the first protease is a metalloprotease. In one embodiment, the first protease is a metalloprotease derived from a fungus. In a preferred embodiment, the first protease is derived from the fungus Thermoascus aurantiacus.

In another embodiment, the first protease is a protease from a bacterium of the genus Pyrococcus, preferably derived from Pyrococcus furiosus or a variant thereof. In one embodiment, the first protease is a commercial protease. In one embodiment, the first protease is Pfu Protease S from Takara Bio Inc.

Other suitable commercial proteases include SZM AP-1+, FP Elite and TSP+ available from CTE Global Inc.

In one embodiment, the composition comprising the second alpha-amylase and the first protease is a commercially available enzyme blend for starch processing. Suitable commercially available enzyme blends include, but are not limited to, BEX 10,000 and COMBO® available from American Biosystems; Avantec® and Avantec® Amp available from Novozymes; AMYL-LTP+ and AMYL-XTP+ available from CTE Global Inc. In a preferred embodiment, the composition comprising the second alpha-amylase and the first protease is Avantec® Amp available from Novozymes.

In one embodiment, the composition comprising the second alpha-amylase and the first protease is added at an amount of about 0.001% to about 0.01% weight of enzyme per weight of raw plant material. In one embodiment, the composition comprising the second alpha-amylase and the first protease is added at an amount of about 0.002% to about 0.01%, about 0.003% to about 0.01%, about 0.004% to about 0.01%, about 0.005% to about 0.01%, about 0.006% to about 0.01%, about 0.007% to about 0.01%, about 0.008% to about 0.01%, about 0.009% to about 0.01%, about 0.001% to about 0.009%, about 0.002% to about 0.009%, about 0.003% to about 0.009%, about 0.004% to about 0.009%, about 0.005% to about 0.009%, about 0.006% to about 0.009%, about 0.007% to about 0.009%, about 0.008% to about 0.009%, about 0.001% to about 0.008%, about 0.002% to about 0.008%, about 0.003% to about 0.008%, about 0.004% to about 0.008%, about 0.005% to about 0.008%, about 0.006% to about 0.008%, about 0.007% to about 0.008%, about 0.001% to about 0.007%, about 0.002% to about 0.007%, about 0.003% to about 0.007%, about 0.004% to about 0.007%, about 0.005% to about 0.007%, about 0.006% to about 0.007%, about 0.001% to about 0.006%, about 0.002% to about 0.006%, about 0.003% to about 0.006%, about 0.004% to about 0.006%, about 0.005% to about 0.006%, about 0.001% to about 0.005%, about 0.002% to about 0.005%, about 0.003% to about 0.005%, about 0.004% to about 0.005%, about 0.001% to about 0.004%, about 0.002% to about 0.004%, about 0.003% to about 0.004%, about 0.001% to about 0.003%, about 0.002% to about 0.003%, 0.001% to about 0.002%, weight of enzyme per weight of raw plant material. In a preferred embodiment, the composition comprising the second alpha-amylase and the first protease is added at an amount of about 0.004% to 0.005% weight of enzyme per weight of raw plant material.

In another embodiment, the composition comprising the second alpha-amylase and the first protease is added at an amount of about 0.001%, about 0.002%, about 0.003%, about 0.004%, about 0.005%, about 0.006%, about 0.007%, about 0.008%, about 0.009% or about 0.01% weight of enzyme per weight of raw plant material. In a preferred embodiment, the composition comprising the second alpha-amylase and the first protease is added at an amount of about 0.005% weight of enzyme per weight of raw plant material.

In one embodiment, the first alpha-amylase and the composition comprising the second alpha-amylase and the first protease are added simultaneously. “Simultaneously” means that the first alpha-amylase and composition comprising the second alpha-amylase and the first protease are added at the same time. In one embodiment, the first alpha-amylase and the composition comprising the second alpha-amylase and the first protease are added separately and simultaneously, i.e. the first alpha-amylase is added as a first solution and the composition comprising the second alpha-amylase and the first protease is added to the slurry at the same time, but as part of a second solution. The first alpha-amylase and the composition comprising the second alpha-amylase and the first protease may be added from separate containers. The first alpha-amylase and the composition comprising the second alpha-amylase and the first protease may be pre-mixed or blended before being added simultaneously.

In another embodiment, the first alpha-amylase and the composition comprising the second alpha-amylase and the first protease are added separately. The first alpha-amylase and the composition comprising the second alpha-amylase and the first protease may be added one after the other. The first alpha-amylase and the composition comprising the second alpha-amylase and the first protease may be added at different stages of the process. The first alpha-amylase and the second alpha-amylase may be added up to 90 minutes apart. In one embodiment, the first alpha-amylase and the second alpha-amylase are added 5 minutes apart, between 5 and 10 minutes apart, between 10 and 20 minutes apart, between 20 and 30 minutes apart, between 30 and 40 minutes apart, between 40 and 50 minutes apart, between 50 and 60 minutes apart, between 60 and 70 minutes apart, between 70 and 80 minutes apart or between 80 and 90 minutes apart.

In one embodiment, the first alpha-amylase is added in an amount of about 0.01% to about 0.06% weight of enzyme per weight of raw plant material. In another embodiment, the first alpha-amylase is added in an amount of about 0.01% to about 0.055%, about 0.01% to about 0.05%, about 0.01% to about 0.045%, about 0.01% to about 0.04%, about 0.01% to about 0.035%, about 0.01% to about 0.03%, about 0.01% to about 0.025%, about 0.01% to about 0.02%, about 0.01% to about 0.015%, about 0.015% to about 0.06%, about 0.015% to about 0.055%, about 0.015% to about 0.05%, about 0.015% to about 0.045%, about 0.015% to about 0.04%, about 0.015% to about 0.035%, about 0.015% to about 0.03%, about 0.015% to about 0.025%, about 0.015% to about 0.02%, about 0.02% to about 0.06%, about 0.02% to about 0.055%, about 0.02% to about 0.05%, about 0.02% to about 0.045%, about 0.02% to about 0.04%, about 0.02% to about 0.035%, about 0.02% to about 0.03%, about 0.02% to about 0.025%, about 0.025% to about 0.06%, about 0.025% to about 0.055%, about 0.025% to about 0.05%, about 0.025% to about 0.045%, about 0.025% to about 0.04%, about 0.025% to about 0.035%, about 0.025% to about 0.03%, about 0.03% to about 0.06%, about 0.03% to about 0.055%, about 0.03% to about 0.05%, about 0.03% to about 0.045%, about 0.03% to about 0.04%, about 0.03% to about 0.035%, about 0.035% to about 0.06%, about 0.035% to about 0.055%, about 0.035% to about 0.05%, about 0.035% to about 0.045%, about 0.035% to about 0.04%, about 0.04% to about 0.06%, about 0.04% to about 0.055%, about 0.04% to about 0.05%, about 0.04% to about 0.045%, about 0.045% to about 0.06%, about 0.045% to about 0.055%, about 0.045% to about 0.05%, about 0.05% to about 0.06%, about 0.05% to about 0.055%, or about 0.055% to about 0.06% weight of enzyme per weight of raw plant material. In a preferred embodiment, the first alpha-amylase is added at an amount of about 0.027% to 0.04% weight of enzyme per weight of raw plant material.

In another embodiment, the first alpha-amylase is added in an amount of about 0.01%, 0.011%, about 0.012%, about 0.013% about 0.014%, about 0.015%, about 0.016%, about 0.017%, about 0.018%, about 0.019%, about 0.02%, 0.021%, about 0.022%, about 0.023% about 0.024%, about 0.025%, about 0.026%, about 0.027%, about 0.028%, about 0.029%, about 0.03%, 0.031%, about 0.032%, about 0.033% about 0.034%, about 0.035%, about 0.036%, about 0.037%, about 0.038%, about 0.039%, about 0.04%, 0.041%, about 0.042%, about 0.043% about 0.044%, about 0.045%, about 0.046%, about 0.047%, about 0.048%, about 0.049%, about 0.05%, 0.051%, about 0.052%, about 0.053% about 0.054%, about 0.055%, about 0.056%, about 0.057%, about 0.058%, about 0.059%, or about 0.06% weight of enzyme per weight of raw plant material. In a preferred embodiment, the first alpha-amylase is added in an amount of about 0.027% weight of enzyme per weight of raw plant material.

In one embodiment, the first alpha-amylase is added in an amount of about 0.27% weight of enzyme per weight of raw plant material and the composition comprising the second alpha-amylase and the first protease is added at an amount of about 0.005% weight of enzyme per weight of raw plant material.

### Additional Enzymes

In one embodiment, the method of starch processing further comprises adding a second protease and/or a glucoamylase.

In one embodiment, the second protease is an aspartic endopeptidase. In one embodiment, the second protease is an aspartic protease from Aspergillus niger or a variant thereof. In one embodiment, the second protease is an aspergillopeptidase.

The term “glucoamylase” refers to enzymes from the family of gamma-amylases (4-alpha-D-glucan glucohydrolase, EC 3.2.1.3), which can rapidly hydrolyze 1→6-alpha-D-glucosidic bonds when the next bond in the sequence is 1→4, and some preparations of this enzyme hydrolyze 1→6- and 1→3-alpha-D-glucosidic bonds in other polysaccharides. In starch processing, the enzyme hydrolyses terminal (1→4)-linked alpha-D-glucose residues successively from non-reducing ends of the chains of amylose of amylopectin with release of beta-D-glucose.

Glucoamylases are capable of producing a carbohydrate that can be used as an energy-source by the fermenting organism(s) in question, for instance, when used in a process for producing a fermentation product, such as ethanol. Glucoamylases can be used in laboratory and industrial settings to hydrolyze a polysaccharide, oligosaccharide or starch, or any maltodextrin-comprising compound for a variety of purposes. These glucoamylases can be used alone to provide specific hydrolysis or can be combined with other glucoamylases to provide a “cocktail” with a broad spectrum of activity. Exemplary uses include the removal or partial or complete hydrolysis of a polysaccharide, oligosaccharide or starch, or any maltodextrin-comprising compound from biological, food, animal feed, pharmaceutical or industrial samples.

In one embodiment, the glucoamylase is a thermostable glucoamylase. According to the invention, the glucoamylase present during starch processing may be derived from any suitable source, e.g. derived from a microorganism, animal or a plant. Preferred glucoamylases are derived from fungi or bacteria. In one embodiment, glucoamylase is derived from the genus Aspergillus, in particular, the glucoamylase is derived from Aspergillus niger or Aspergillus oryzae. Suitable glucoamylases may also be derived from microorgansims from any of the genera Athelia, Talaromyces, Clostridium, Trametes, Trichoderma, Pachykytospora, Rhyzopus, Leucopaxillus, Penicillium, Peniophora, Pycnoporus, Gloephyllum, Nigroformes or Bacillus.

In one embodiment, the composition comprising a glucoamylase comprises a primary glucoamylase and a secondary glucoamylase. In one embodiment, the secondary glucoamylase is a glucoamylase with debranching activity. In one embodiment, the composition comprising a glucoamylase additionally comprises a glucoamylase with debranching activity, a fungal alpha-amylase and a trehalase.

Commercially available compositions comprising glucoamylase include AMG 200L, AMG 300L, SAN™ SUPER, SAN™ EXTRA L, SPIRIZYME™ PLUS, SPIRIZYME™ ULTRA, SPIRIZYME™ ECXEL, Extenda® and AMG™ E from Novozymes A/S; OPTIDEX™ 300, DISTILLASE, GC480, GC417, G-ZYME™ G900, G-ZYME™ and G990 ZR from Genencor Int.; AMIGASE™ and AMIGASE™ PLUS from DSM, Supreme Extreme, Strive CR, Strive EX, Strive May, Supreme Max, LG Prime, Glucoamyl L-209+ and Endure X from CTE Global Inc. In a preferred embodiment, the composition comprising a glucoamylase is Supreme Extreme available from CTE Global Inc.

The method of the invention may comprise addition of other enzymes, such as additional amylases, alpha-, beta-, and gamma-amylase, additional protease, debranching enzymes, pullulanase, maltase, glucose isomerase, dextranase, lipase, pentosanase, alpha-acetolactate decarboxylase, xylanase, beta-glucanase, trehalase, and cellulase.

In one embodiment, the method of starch processing comprises addition of the first alpha-amylase according to SEQ ID NO: 1, Avantec® Amp available from Novozymes and Supreme Extreme available from CTE Global Inc.

### Methods of the Invention

In one embodiment, the method of starch processing comprises the step of adding the first alpha-amylase and the second alpha-amylase to a slurry comprising a starch, thereby forming a mixture and incubating said mixture.

The term “mixture” refers to a new composite substance made from a combination of different substances through the process of incorporating them into the new composite substance.

Incubation of the mixture of the first and second alpha-amylase and the slurry comprising a starch in the slurry mix tank is the first step of the process.

In one embodiment, the mixture is incubated for a period of about 10 minutes to about 60 minutes, about 10 minutes to about 55 minutes, about 10 minutes to about 50 minutes, about 10 minutes to about 45 minutes, about 10 minutes to about 40 minutes, about 10 minutes to about 35 minutes, about 10 minutes to about 30 minutes, about 10 minutes to about 25 minutes, about 10 minutes to about 20 minutes, about 10 minutes to about 15 minutes, 15 minutes to about 60 minutes, about 15 minutes to about 55 minutes, about 15 minutes to about 50 minutes, about 15 minutes to about 45 minutes, about 15 minutes to about 40 minutes, about 15 minutes to about 35 minutes, about 15 minutes to about 30 minutes, about 15 minutes to about 25 minutes, about 15 minutes to about 20 minutes, about 20 minutes to about 60 minutes, about 20 minutes to about 55 minutes, about 20 minutes to about 50 minutes, about 20 minutes to about 45 minutes, about 20 minutes to about 40 minutes, about 20 minutes to about 35 minutes, about 20 minutes to about 30 minutes, about 20 minutes to about 25 minutes, about 25 minutes to about 60 minutes, about 25 minutes to about 55 minutes, about 25 minutes to about 50 minutes, about 25 minutes to about 45 minutes, about 25 minutes to about 40 minutes, about 25 minutes to about 35 minutes, about 25 minutes to about 30 minutes, about 30 minutes to about 60 minutes, about 30 minutes to about 55 minutes, about 30 minutes to about 50 minutes, about 30 minutes to about 45 minutes, about 30 minutes to about 40 minutes, about 30 minutes to about 35 minutes, about 35 minutes to about 60 minutes, about 35 minutes to about 55 minutes, about 35 minutes to about 50 minutes, about 35 minutes to about 45 minutes, about 35 minutes to about 40 minutes, about 40 minutes to about 60 minutes, about 40 minutes to about 55 minutes, about 40 minutes to about 50 minutes, about 40 minutes to about 45 minutes, about 45 minutes to about 60 minutes, about 45 minutes to about 55 minutes, about 45 minutes to about 50 minutes, about 50 minutes to about 60 minutes, about 50 minutes to about 55 minutes, about 55 minutes to about 60 minutes after addition of the of the first and second alpha-amylase to the slurry tank.

In a preferred embodiment, the mixture is incubated for about 10 minutes to about 30 minutes after addition of the first and second alpha-amylase to the slurry tank 15 minutes to about 25 minutes after addition of the first and second alpha-amylase to the slurry tank.

In another embodiment, the mixture is incubated for about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes after addition of the enzymes to the slurry tank. In a preferred embodiment, the mixture is incubated for about 18 minutes after addition of the first and second alpha-amylase to the slurry tank.

The present invention is also directed to a method of starch processing further comprising adding the first alpha-amylase and the second alpha-amylase during a liquefying stage. The term “liquefying stage” or “liquefaction” refers to the stage in starch conversion in which gelatinized starch is hydrolyzed to give low molecular weight soluble sugars.

In one embodiment, the method of starch processing comprises the step of adding the first alpha-amylase and the second alpha-amylase to a slurry having a starch during a liquefying stage, thereby forming a mixture and incubating said mixture. Once in the liquefaction tank, the mixture is incubated for a second, longer time period than in the slurry tank.

In one embodiment, said mixture is incubated for a period of about 60 minutes to about 210 minutes, about 60 minutes to about 180 minutes, about 60 minutes to about 150 minutes, about 60 minutes to about 120 minutes, about 60 minutes to about 90 minutes, about 90 minutes to about 210 minutes, about 90 minutes to about 180 minutes, about 90 minutes to about 150 minutes, about 90 minutes to about 120 minutes, about 120 minutes to about 210 minutes, about 120 minutes to about 150 minutes, about 120 minutes to about 180 minutes, about 150 minutes to about 180 minutes, about 150 minutes to about 210 minutes, about 180 minutes to about 210 minutes during a liquefying stage. In a preferred embodiment, the mixture is incubated for a period of about 120 minutes to about 150 minutes during a liquefying stage.

In another embodiment, the mixture is incubated for a period of about 60 minutes, about 70 minutes, about 80 minutes, about 90 minutes, about 100 minutes, about 110 minutes, about 120 minutes, about 130 minutes, about 140 minutes, about 150 minutes, 160 minutes, about 170 minutes, about 180 minutes, about 190 minutes, about 200 minutes, or about 210 minutes during a liquefying stage. In a preferred embodiment, the mixture is incubated for a period of about 150 minutes during a liquefying stage.

In one embodiment, the method of the invention is performed at a temperature of about 62° C. to about 95° C., about 65° C. to about 95° C., about 70° C. to about 95° C., about 75° C. to about 95° C., about 80° C. to about 95° C., about 85° C. to about 95° C., about 90° C. to about 95%, about 62° C. to about 90° C., about 65° C. to about 90° C., about 70° C. to about 90° C., about 75° C. to about 90° C., about 80° C. to about 90° C., about 85° C. to about 90° C., about 62° C. to about 85° C., about 65° C. to about 85° C., about 70° C. to about 85° C., about 75° C. to about 85° C., about 80° C. to about 85° C., about 62° C. to about 80° C., about 65° C. to about 80° C., about 70° C. to about 80° C., about 75° C. to about 80° C., about 62° C. to about 75° C., about 65° C. to about 75° C., about 70° C. to about 75° C., about 62° C. to about 70° C., about 65° C. to about 70° C., or about 62° C. to about 65° C. In a preferred embodiment, the method of the invention is performed at a temperature of about 75° C. to about 90° C. In a particularly preferred embodiment, the method of the invention is performed at a temperature of about 80° C. to about 95° C.

In another embodiment, the method of the invention is performed at a temperature of about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., or about 95° C. In a preferred embodiment, the method of the invention is performed at a temperature of about 88° C.

In one embodiment, the method of the present invention is performed at a pH of 4.3 to 6.5. In another embodiment, the method of the present invention is performed at a pH of 4.3 to 6.3, a pH of 4.3 to 6.0, a pH of 4.3 to 5.7, a pH of 4.3 to 5.5, a pH of 4.3 to 5.3, a pH of 4.3 to 5.0, a pH of 4.3 to 4.7, a pH of 4.3 to 4.5, a pH of 4.5 to 6.5, a pH of 4.5 to 6.3, a pH of 4.5 to 6.0, a pH of 4.5 to 5.7, a pH of 4.5 to 5.5, a pH of 4.5 to 5.3, a pH of 4.5 to 5.0, a pH of 4.5 to 4.7, a pH of 5.0 to 6.5, a pH of 5.0 to 6.3, a pH of 5.0 to 6.0, a pH of 5.0 to 5.7, a pH of 5.0 to 5.5, a pH of 5.0 to 5.3, a pH of 5.3 to 6.5, a pH of 5.3 to 6.3, a pH of 5.3 to 6.0, a pH of 5.3 to 5.7, a pH of 5.3 to 5.5, a pH of 5.5 to 5.7. In a preferred embodiment, the method of the present invention is performed at a pH of 4.5 to 5.7.

In another embodiment, the method of the present invention is performed at a pH of about 4.3, about 4.5, about 4.7 about 5.0, about 5.2, about 5.5, about 5.7, about 6.0, about 6.2 or about 6.5. In a preferred embodiment, the method of the present invention is performed at a pH of about 5.0.

In one embodiment, the mixture is incubated at a temperature of 75° C. and a pH of 4.5, at a temperature of 75° C. and a pH of 5.0, at a temperature of 75° C. and a pH of 5.5, at a temperature of 75° C. and a pH of 6.0 or at a temperature of 75° C. and a pH of 6.5.

In another embodiment, the mixture is incubated at a temperature of 80° C. and a pH of 4.5, at a temperature of 80° C. and a pH of 5.0, at a temperature of 80° C. and a pH of 5.5, at a temperature of 80° C. and a pH of 6.0 or at a temperature of 80° C. and a pH of 6.5.

In another embodiment, the mixture is incubated at a temperature of 85° C. and a pH of 4.5, at a temperature of 85° C. and a pH of 5.0, at a temperature of 85° C. and a pH of 5.5, at a temperature of 85° C. and a pH of 6.0 or at a temperature of 85° C. and a pH of 6.5.

In another embodiment, the mixture is incubated at a temperature of 90° C. and a pH of 4.5, at a temperature of 90° C. and a pH of 5.0, at a temperature of 90° C. and a pH of 5.5, at a temperature of 90° C. and a pH of 6.0 or at a temperature of 90° C. and a pH of 6.5.

In some embodiments, the first alpha-amylase is present in an amount from about 0.01% weight of enzyme per weight of raw plant material, the weight ratio of the first alpha-amylase to the second alpha-amylase is about 1:2, the mixture is incubated at a temperature of about 80° C. and a pH of 6.0.

In another embodiment, the first alpha-amylase is present in an amount from about 0.01% weight of enzyme per weight of raw plant material, the weight ratio of the first alpha-amylase to the second alpha-amylase is about 1:2 and the mixture is incubated at a temperature of about 85° C. and a pH of 6.0.

In another embodiment, the first alpha-amylase is present in an amount from about 0.01% weight of enzyme per weight of raw plant material, the weight ratio of the first alpha-amylase to the second alpha-amylase is about 1:2 and the mixture is incubated at a temperature of about 90° C. and a pH of 6.0.

In some embodiments, the first alpha-amylase is present in an amount from about 0.01% weight of enzyme per weight of raw plant material, the weight ratio of the first alpha-amylase to the second alpha-amylase is about 1:2, the mixture is incubated at a temperature of about 80° C. and a pH of 5.5.

In another embodiment, the first alpha-amylase is present in an amount from about 0.01% weight of enzyme per weight of raw plant material, the weight ratio of the first alpha-amylase to the second alpha-amylase is about 1:2 and the mixture is incubated at a temperature of about 85° C. and a pH of 5.5.

In another embodiment, the first alpha-amylase is present in an amount from about 0.01% weight of enzyme per weight of raw plant material, the weight ratio of the first alpha-amylase to the second alpha-amylase is about 1:2 and the mixture is incubated at a temperature of about 90° C. and a pH of 5.5.

In some embodiments, the first alpha-amylase is present in an amount from about 0.01% weight of enzyme per weight of raw plant material, the weight ratio of the first alpha-amylase to the second alpha-amylase is about 1:2, the mixture is incubated at a temperature of about 80° C. and a pH of 5.0.

In another embodiment, the first alpha-amylase is present in an amount from about 0.01% weight of enzyme per weight of raw plant material, the weight ratio of the first alpha-amylase to the second alpha-amylase is about 1:2 and the mixture is incubated at a temperature of about 85° C. and a pH of 5.0.

In another embodiment, the first alpha-amylase is present in an amount from about 0.01% weight of enzyme per weight of raw plant material, the weight ratio of the first alpha-amylase to the second alpha-amylase is about 1:2 and the mixture is incubated at a temperature of about 90° C. and a pH of 5.0.

In some embodiments, the weight ratio of the first alpha-amylase to the second alpha-amylase is about 1:5 and the mixture is incubated at a temperature of about 80° C.; the weight ratio of the first alpha-amylase to the second alpha-amylase is about 1:5 and the mixture is incubated at a temperature of about 85° C.; the weight ratio of the first alpha-amylase to the second alpha-amylase is about 1:5 and the mixture is incubated at a temperature of about 90° C.; the weight ratio of the first alpha-amylase to the second alpha-amylase is about 1:10 and the mixture is incubated at a temperature of about 80° C.; the weight ratio of the first alpha-amylase to the second alpha-amylase is about 1:10 and the mixture is incubated at a temperature of about 85° C.; the weight ratio of the first alpha-amylase to the second alpha-amylase is about 1:10 and the mixture is incubated at a temperature of about 90° C., the weight ratio of the first alpha-amylase to the second alpha-amylase is about 2:1 and the mixture is incubated at a temperature of about 80° C.; the weight ratio of the first alpha-amylase to the second alpha-amylase is about 2:1 and the mixture is incubated at a temperature of about 85° C.; the weight ratio of the first alpha-amylase to the second alpha-amylase is about 2:1 and the mixture is incubated at a temperature of about 90° C.; the weight ratio of the first alpha-amylase to the second alpha-amylase is about 5:1 and the mixture is incubated at a temperature of about 80° C.; the weight ratio of the first alpha-amylase to the second alpha-amylase is about 5:1 and the mixture is incubated at a temperature of about 85° C.; or the weight ratio of the first alpha-amylase to the second alpha-amylase is about 5:1 and the mixture is incubated at a temperature of about 90° C.

In some embodiments, the weight ratio of the first alpha-amylase to the second alpha-amylase is about 1:5, the mixture is incubated at a temperature of about 80° C. and a pH of 6.0; the weight ratio of the first alpha-amylase to the second alpha-amylase is about 1:5 and the mixture is incubated at a temperature of about 85° C. and a pH of 6.0; the weight ratio of the first alpha-amylase to the second alpha-amylase is about 1:5 and the mixture is incubated at a temperature of about 90° C. and a pH of 6.0; the weight ratio of the first alpha-amylase to the second alpha-amylase is about 1:10 and the mixture is incubated at a temperature of about 80° C. and a pH of 6.0; the weight ratio of the first alpha-amylase to the second alpha-amylase is about 1:10 and the mixture is incubated at a temperature of about 85° C. and a pH of 6.0; the weight ratio of the first alpha-amylase to the second alpha-amylase is about 1:10 and the mixture is incubated at a temperature of about 90° C. and a pH of 6.0, the weight ratio of the first alpha-amylase to the second alpha-amylase is about 2:1 and the mixture is incubated at a temperature of about 80° C. and a pH of 6.0; the weight ratio of the first alpha-amylase to the second alpha-amylase is about 2:1 and the mixture is incubated at a temperature of about 85° C. and a pH of 6.0; the weight ratio of the first alpha-amylase to the second alpha-amylase is about 2:1 and the mixture is incubated at a temperature of about 90° C. and a pH of 6.0; the weight ratio of the first alpha-amylase to the second alpha-amylase is about 5:1 and the mixture is incubated at a temperature of about 80° C. and a pH of 6.0; the weight ratio of the first alpha-amylase to the second alpha-amylase is about 5:1 and the mixture is incubated at a temperature of about 85° C. and a pH of 6.0; or the weight ratio of the first alpha-amylase to the second alpha-amylase is about 5:1 and the mixture is incubated at a temperature of about 90° C. and a pH of 6.0.

In one embodiment, the first alpha-amylase is present in an amount of about 0.0035% total weight of enzyme by weight of raw plant material and the second alpha-amylase is present in an amount of about 0.002% total weight of enzyme by weight of raw plant material and the mixture is incubated at a temperature of 80° C.

In one embodiment, the first alpha-amylase is present in an amount of about 0.0069% total weight of enzyme by weight of raw plant material and the second alpha-amylase is present in an amount of about 0.0012% total weight of enzyme by weight of raw plant material and the mixture is incubated at a temperature of 80° C.

In one embodiment, the first alpha-amylase is present in an amount of about 0.0019% total weight of enzyme by weight of raw plant material and the second alpha-amylase is present in an amount of about 0.0037% total weight of enzyme by weight of raw plant material and the mixture is incubated at a temperature of 80° C.

In one embodiment, the first alpha-amylase is present in an amount of about 0.0057% total weight of enzyme by weight of raw plant material and the second alpha-amylase is present in an amount of about 0.0044% total weight of enzyme by weight of raw plant material and the mixture is incubated at a temperature of 80° C.

In one embodiment, the first alpha-amylase is present in an amount of about 0.01% total weight of enzyme by weight of raw plant material and the second alpha-amylase is present in an amount of about 0.0054% total weight of enzyme by weight of raw plant material and the mixture is incubated at a temperature of 80° C.

In one embodiment, the first alpha-amylase is present in an amount of about 0.0065% total weight of enzyme by weight of raw plant material and the second alpha-amylase is present in an amount of about 0.0083% total weight of enzyme by weight of raw plant material and the mixture is incubated at a temperature of 80° C.

In one embodiment, the first alpha-amylase is present in an amount of about 0.003% total weight of enzyme by weight of raw plant material and the second alpha-amylase is present in an amount of about 0.01% total weight of enzyme by weight of raw plant material and the mixture is incubated at a temperature of 80° C.

In one embodiment, the first alpha-amylase is present in an amount of about 0.01% total weight of enzyme by weight of raw plant material and the second alpha-amylase is present in an amount of about 0.01% total weight of enzyme by weight of raw plant material and the mixture is incubated at a temperature of 80° C.

In one embodiment, the first alpha-amylase is present in an amount of about 0.009% total weight of enzyme by weight of raw plant material and the second alpha-amylase is present in an amount of about 0.001% total weight of enzyme by weight of raw plant material and the mixture is incubated at a temperature of 80° C.

In one embodiment, the first alpha-amylase is present in an amount of about 0.009% total weight of enzyme by weight of raw plant material and the second alpha-amylase is present in an amount of about 0.002% total weight of enzyme by weight of raw plant material and the mixture is incubated at a temperature of 80° C.

In one embodiment, the first alpha-amylase is present in an amount of about 0.007% total weight of enzyme by weight of raw plant material and the second alpha-amylase is present in an amount of about 0.003% total weight of enzyme by weight of raw plant material and the mixture is incubated at a temperature of 80° C.

In one embodiment, the first alpha-amylase is present in an amount of about 0.0035% total weight of enzyme by weight of raw plant material and the second alpha-amylase is present in an amount of about 0.002% total weight of enzyme by weight of raw plant material and the mixture is incubated at a temperature of 85° C.

In one embodiment, the first alpha-amylase is present in an amount of about 0.0069% total weight of enzyme by weight of raw plant material and the second alpha-amylase is present in an amount of about 0.0012% total weight of enzyme by weight of raw plant material and the mixture is incubated at a temperature of 85° C.

In one embodiment, the first alpha-amylase is present in an amount of about 0.0019% total weight of enzyme by weight of raw plant material and the second alpha-amylase is present in an amount of about 0.0037% total weight of enzyme by weight of raw plant material and the mixture is incubated at a temperature of 85° C.

In one embodiment, the first alpha-amylase is present in an amount of about 0.0057% total weight of enzyme by weight of raw plant material and the second alpha-amylase is present in an amount of about 0.0044% total weight of enzyme by weight of raw plant material and the mixture is incubated at a temperature of 85° C.

In one embodiment, the first alpha-amylase is present in an amount of about 0.01% total weight of enzyme by weight of raw plant material and the second alpha-amylase is present in an amount of about 0.0054% total weight of enzyme by weight of raw plant material and the mixture is incubated at a temperature of 85° C.

In one embodiment, the first alpha-amylase is present in an amount of about 0.0065% total weight of enzyme by weight of raw plant material and the second alpha-amylase is present in an amount of about 0.0083% total weight of enzyme by weight of raw plant material and the mixture is incubated at a temperature of 85° C.

In one embodiment, the first alpha-amylase is present in an amount of about 0.003% total weight of enzyme by weight of raw plant material and the second alpha-amylase is present in an amount of about 0.01% total weight of enzyme by weight of raw plant material and the mixture is incubated at a temperature of 85° C.

In one embodiment, the first alpha-amylase is present in an amount of about 0.01% total weight of enzyme by weight of raw plant material and the second alpha-amylase is present in an amount of about 0.01% total weight of enzyme by weight of raw plant material and the mixture is incubated at a temperature of 85° C.

In one embodiment, the first alpha-amylase is present in an amount of about 0.009% total weight of enzyme by weight of raw plant material and the second alpha-amylase is present in an amount of about 0.001% total weight of enzyme by weight of raw plant material and the mixture is incubated at a temperature of 85° C.

In one embodiment, the first alpha-amylase is present in an amount of about 0.009% total weight of enzyme by weight of raw plant material and the second alpha-amylase is present in an amount of about 0.002% total weight of enzyme by weight of raw plant material and the mixture is incubated at a temperature of 85° C.

In one embodiment, the first alpha-amylase is present in an amount of about 0.007% total weight of enzyme by weight of raw plant material and the second alpha-amylase is present in an amount of about 0.003% total weight of enzyme by weight of raw plant material and the mixture is incubated at a temperature of 85° C.

In one embodiment, the first alpha-amylase is present in an amount of about 0.0035% total weight of enzyme by weight of raw plant material and the second alpha-amylase is present in an amount of about 0.002% total weight of enzyme by weight of raw plant material and the mixture is incubated at a temperature of 90° C.

In one embodiment, the first alpha-amylase is present in an amount of about 0.0069% total weight of enzyme by weight of raw plant material and the second alpha-amylase is present in an amount of about 0.0012% total weight of enzyme by weight of raw plant material and the mixture is incubated at a temperature of 90° C.

In one embodiment, the first alpha-amylase is present in an amount of about 0.0019% total weight of enzyme by weight of raw plant material and the second alpha-amylase is present in an amount of about 0.0037% total weight of enzyme by weight of raw plant material and the mixture is incubated at a temperature of 90° C.

In one embodiment, the first alpha-amylase is present in an amount of about 0.0057% total weight of enzyme by weight of raw plant material and the second alpha-amylase is present in an amount of about 0.0044% total weight of enzyme by weight of raw plant material and the mixture is incubated at a temperature of 90° C.

In one embodiment, the first alpha-amylase is present in an amount of about 0.01% total weight of enzyme by weight of raw plant material and the second alpha-amylase is present in an amount of about 0.0054% total weight of enzyme by weight of raw plant material and the mixture is incubated at a temperature of 90° C.

In one embodiment, the first alpha-amylase is present in an amount of about 0.0065% total weight of enzyme by weight of raw plant material and the second alpha-amylase is present in an amount of about 0.0083% total weight of enzyme by weight of raw plant material and the mixture is incubated at a temperature of 90° C.

In one embodiment, the first alpha-amylase is present in an amount of about 0.003% total weight of enzyme by weight of raw plant material and the second alpha-amylase is present in an amount of about 0.01% total weight of enzyme by weight of raw plant material and the mixture is incubated at a temperature of 90° C.

In one embodiment, the first alpha-amylase is present in an amount of about 0.01% total weight of enzyme by weight of raw plant material and the second alpha-amylase is present in an amount of about 0.01% total weight of enzyme by weight of raw plant material and the mixture is incubated at a temperature of 90° C.

In one embodiment, the first alpha-amylase is present in an amount of about 0.009% total weight of enzyme by weight of raw plant material and the second alpha-amylase is present in an amount of about 0.001% total weight of enzyme by weight of raw plant material and the mixture is incubated at a temperature of 90° C.

In one embodiment, the first alpha-amylase is present in an amount of about 0.009% total weight of enzyme by weight of raw plant material and the second alpha-amylase is present in an amount of about 0.002% total weight of enzyme by weight of raw plant material and the mixture is incubated at a temperature of 90° C.

In one embodiment, the first alpha-amylase is present in an amount of about 0.007% total weight of enzyme by weight of raw plant material and the second alpha-amylase is present in an amount of about 0.003% total weight of enzyme by weight of raw plant material and the mixture is incubated at a temperature of 90° C.

The term “viscosity” describes a measurement of the resistance of a fluid to deformation at a given rate. When a force is applied to a liquid, it is opposed by internal friction arising from the cohesion of the molecules. This internal friction is the property of a liquid called viscosity.

The skilled person is aware that the factors that can influence the viscosity of the slurry include the percentage of solid fraction, the shape and size distribution of particles, and temperature. Viscosity can be measured with a viscometer and/or rheometer. Suitable viscometers include glass capillary viscometers, falling-sphere or piston viscometers, oscillating or vibrational viscometers, and rotational viscometers. In one embodiment, the viscosity is measured using a Brookfield LV rotational viscometer with LV-2 spindle at room temperature.

In one embodiment, the viscosity of the slurry is reduced compared to a slurry not comprising the second alpha-amylase.

In one embodiment, the viscosity of the slurry is reduced by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70% compared to a slurry not comprising the second alpha-amylase. In a preferred embodiment, the viscosity of the slurry is reduced by at least 35% compared to a slurry not comprising the second alpha-amylase.

In one embodiment, the viscosity of the slurry is reduced by about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 70%, about 30% to about 65%, about 30% to about 60%, about 30% to about 55%, about 30% to about 50%, about 30% to about 45%, about 30% to about 40%, about 30% to about 35%, about 35% to about 70%, about 35% to about 65%, about 35% to about 60%, about 35% to about 55%, about 35% to about 50%, about 35% to about 45%, about 35% to about 40%, about 40% to about 70%, about 40% to about 65%, about 40% to about 60%, about 40% to about 55%, about 40% to about 50%, about 40% to about 45%, about 45% to about 70%, about 45% to about 65%, about 45% to about 60%, about 45% to about 55%, about 45% to about 50%, about 50% to about 70%, about 50% to about 65%, about 50% to about 60%, about 50% to about 55%, about 55% to about 70%, about 55% to about 65%, about 55% to about 60%, about 60% to about 70%, about 60% to about 65%, about 65% to about 70% compared to a slurry not comprising the second alpha-amylase. In a preferred embodiment, the viscosity of the slurry is reduced by about 30% to about 40% compared to a slurry not comprising the second alpha-amylase.

The term “dextrose equivalent” or “DE” is used to indicate the degree of hydrolysis of starch into glucose in a slurry, expressed in percentage of dry starting material. The higher the DE, the more starch has been converted into glucose.

The term “solids” refers to the total dissolved solids and the total suspended solids in a slurry. Above a certain amount of solids, the slurry is very difficult to treat and can be responsible for blocking processing equipment. Therefore in current processes, starch slurry to be liquefied typically has up to 40 weight/weight % (w/w %) solids.

The raw plant material, such as whole grains, may be reduced in particle size, e.g., by milling, in order to open up the structure and allowing for further processing. The particle size is reduced to between 0.05 to 3.0 mm, (e.g., 0.05, 0.1, 0.2, 0.3, 0.4, 0.5, 0.6, 0.7, 0.8, 0.9, 1.0, 1.3, 1.5, 1.8, 2.0, 2.3, 2.5, 2.7, 3.0, and ranges in between). Particle size is determined by sifting the solids through screens with varying mesh and then weighing each fraction.

It is contemplated that methods disclosed herein can be used in conjunction with any milling technique employed to process the raw plant material before incubation in the slurry tank, including, but not limited to, wet milling, dry milling, dry grinding, cracking, coarse grinding, fine grinding, fractionating, mixing, flaking, steam flaking, rolling or chopping.

The concentration of solids in the slurry is measured as a dry weight percentage by taking a process sample and analyzing the sample in a moisture balance and/or in process density meter. In one embodiment, the slurry comprises 20% to 45%, 20% to 42%, 20% to 40%, 20% to 37%, 20% to 35%, 20% to 32%, 20% to 30%, 20% to 27%, 20% to 25%, 20% to 22%, 22% to 45%, 22% to 42%, 22% to 40%, 22% to 37%, 22% to 35%, 22% to 32%, 22% to 30%, 22% to 27%, 22% to 25%, 25% to 45%, 25% to 42%, 25% to 40%, 25% to 37%, 25% to 35%, 25% to 32%, 25% to 30%, 25% to 27%, 27% to 45%, 27% to 42%, 27% to 40%, 27% to 37%, 27% to 35%, 27% to 32%, 27% to 30%, 30% to 45%, 30% to 42%, 30% to 40%, 30% to 37%, 30% to 35%, 30% to 32%, 32% to 45%, 32% to 42%, 32% to 40%, 32% to 37%, 32% to 35%, 35% to 45%, 35% to 42%, 35% to 40%, 35% to 37%, 37% to 45%, 37% to 42%, 37% to 40%, 40% to 45%, 40% to 42%, or 42% to 45% solids. In a preferred embodiment, the slurry comprises 32% to 40% solids.

In another embodiment, the slurry comprises about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45% solids. In a preferred embodiment, the slurry comprises about 35% solids.

In one embodiment, the slurry comprises about 32% solids, the total enzyme dose is 0.02% weight of enzyme per weight of raw plant material, the second alpha-amylase is present at an inclusion rate of 15% and the viscosity is reduced by at least 35% compared to a slurry not comprising the second alpha-amylase.

In another embodiment, the slurry comprises about 35% solids, the total enzyme dose is 0.03% weight of enzyme per weight of raw plant material, the second alpha-amylase is present at an inclusion rate of 15% and the viscosity is reduced by at least 32% compared to a slurry not comprising the second alpha-amylase.

In one embodiment, the first alpha-amylase is present in an amount from about 0.01% to about 0.03% weight of enzyme per weight of raw plant material and the second alpha-amylase is present at an inclusion rate of 15% to 50%, the mixture is incubated at a temperature of 75° C. and a pH of 5.5 and the slurry comprises about 35% solids.

In another embodiment, the first alpha-amylase is present in an amount from about 0.01% to about 0.03% weight of enzyme per weight of raw plant material and the second alpha-amylase is present at an inclusion rate of 15% to 50%, the mixture is incubated at a temperature of 80° C. and a pH of 5.5 and the slurry comprises about 35% solids.

In another embodiment, the first alpha-amylase is present in an amount from about 0.01% to about 0.03% weight of enzyme per weight of raw plant material and the second alpha-amylase is present at an inclusion rate of 15% to 50% and the mixture is incubated at a temperature of 85° C. and a pH of 5.5 and the slurry comprises about 35% solids.

In one embodiment, the second alpha-amylase is present at an inclusion rate of about 5%, the total enzyme dose is 0.02% weight of enzyme per weight of raw plant material and the slurry comprises about 30% solids.

In one embodiment, the second alpha-amylase is present at an inclusion rate of about 10%, the total enzyme dose is 0.02% weight of enzyme per weight of raw plant material and the slurry comprises about 30% solids.

In one embodiment, the second alpha-amylase is present at an inclusion rate of about 15%, the total enzyme dose is 0.02% weight of enzyme per weight of raw plant material and the slurry comprises about 30% solids.

In one embodiment, the second alpha-amylase is present at an inclusion rate of about 20%, the total enzyme dose is 0.02% weight of enzyme per weight of raw plant material and the slurry comprises about 30% solids.

In another embodiment, the second alpha-amylase is present at an inclusion rate of about 5%, the total enzyme dose is 0.03% weight of enzyme per weight of raw plant material and the slurry comprises about 35% solids.

In another embodiment, the second alpha-amylase is present at an inclusion rate of about 10%, the total enzyme dose is 0.03% weight of enzyme per weight of raw plant material and the slurry comprises about 35% solids.

In another embodiment, the second alpha-amylase is present at an inclusion rate of about 15%, the total enzyme dose is 0.03% weight of enzyme per weight of raw plant material and the slurry comprises about 35% solids.

In another embodiment, the second alpha-amylase is present at an inclusion rate of about 20%, the total enzyme dose is 0.03% weight of enzyme per weight of raw plant material and the slurry comprises about 35% solids.

In one embodiment, the first alpha-amylase is present in an amount from about 0.01% to about 0.04% weight of enzyme per weight of raw plant material and the second alpha-amylase is present at an inclusion rate of 5% to 20%, the mixture is incubated at a temperature of 80° C. and the slurry comprises about 35% solids.

In another embodiment, the second alpha-amylase is present at an inclusion rate of about 5%, the total enzyme dose is 0.02% weight of enzyme per weight of raw plant material and the slurry comprises about 35% solids.

In another embodiment, the second alpha-amylase is present at an inclusion rate of about 10%, the total enzyme dose is 0.02% weight of enzyme per weight of raw plant material and the slurry comprises about 35% solids.

In another embodiment, the second alpha-amylase is present at an inclusion rate of about 15%, the total enzyme dose is 0.02% weight of enzyme per weight of raw plant material and the slurry comprises about 35% solids.

In another embodiment, the second alpha-amylase is present at an inclusion rate of about 20%, the total enzyme dose is 0.02% weight of enzyme per weight of raw plant material and the slurry comprises about 35% solids.

In one embodiment, the method of starch processing of the invention further comprises saccharifying and fermenting the slurry to produce a fermentation product comprising alcohols.

As used herein, the term “saccharifying” refers to enzymatic conversion of liquefied starch from the liquefaction process to glucose or other low molecular weight polysaccharides. At this stage of the process, additional enzymes may be added to the mixture.

The term “fermenting” refers to a process which converts sugars, such as glucose, into cellular energy under anaerobic conditions, producing ATP, alcohol and CO2. A “fermentation product” is one of the products of the fermentation process. An “alcohol” fermentation product refers to one of the by-products of alcoholic fermentation.

Fermentation takes place in a vessel or container that allows CO2 to escape. Suitable vessels or containers are known to the skilled person and include conical or cylindroconical vessels, closed or open vessels, vats or tanks, typically made from stainless steel or other metals, plastic, stone or wood. The skilled person is aware of fermentation methods including warm and cool fermentation. Warm fermentation takes place at between 15° C. and 35° C., typically between 15° C. and 24° C. Cool fermentation takes place at temperatures around 10° C.

The skilled person is aware of additional steps that may occur during, before or after primary fermentation, such as kräusening, lagering, secondary fermentation, tertiary fermentation, bottle fermentation, cask conditioning, barrel-ageing or distilling.

In one embodiment, the method of the invention further comprises recovering a fermentation product. In one embodiment, the fermentation product is alcohol. In another embodiment, the fermentation product is ethanol.

The skilled person is aware of methods of recovering a fermentation including filtering, distilling, bottling, isolation or removal into a suitable container. Suitable filters include sheet filters for rough, fine or sterile filtering or powder medium filters comprising diatomaceous earth or perlite. Recovery of the fermentation product may also include corks, caps, screwcaps, synthetic corks, glass, metal, wood and plastic packaging.

Microorganisms that can perform alcoholic fermentation are known to the skilled person and include fungi (molds), yeasts and bacteria, most commonly the yeast Saccharomyces cerevisiae, or other microorganisms from the genera Saccharomyces, Schizosaccharomyces, Saccharomycopsis, Saccharomycodes, Zygosaccharomyces, Kloeckera, Candida, Hanseniaspora, Torulaspora, Metschnikowia, Zymomonas, and Aspergillus. In a preferred embodiment, the fermentation uses a microorganism from the genus Saccharomyces.

In one embodiment, the ethanol yield of the method comprising the slurry comprising the first alpha-amylase and the composition comprising the second alpha-amylase and the protease is increased compared to a method comprising a slurry not comprising the first alpha-amylase. The term “ethanol yield” refers to the amount of ethanol produced using the methods of the present invention. The concentration of ethanol can be determined by using a flow meter to measure the amount of ethanol and comparing that to the amount of corn being ground (bushels of corn being ground).

In some embodiments, the term refers to the volume of ethanol and in other embodiment the term refers to the concentration of ethanol. Ethanol yield is generally defined as gallons of ethanol produced per bushel of corn input. A bushel of corn is defined as about 56 lb or about 25.4 kg and a gallon of ethanol is about 3.785 l so that the ethanol yield can also be defined as l/kg, which can be calculated from gallons per bushel by multiplying with 0.15. Fermentation ethanol yield can be estimated by % concentration of ethanol divided by the % solids concentration of the mash that fills the fermenter. This is commonly referred to as fermentation ethanol yield ratio.

In one embodiment, the ethanol yield of the method using the first alpha-amylase and the composition comprising the second alpha-amylase and the protease is increased by 0.5% to 5%, by 0.5% to 5%, by 0.6% to 5%, by 0.7% to 5%, by 0.8% to 5%, by 0.9% to 5%, by 1% to 5%, by 1.1% to 5%, by 1.2% to 5%, by 1.3% to 5%, by 1.4% to 5%, by 1.5% to 5%, by 1.6% to 5%, by 1.7% to 5%, by 1.8% to 5%, by 1.9% to 5%, by 2% to 5%, by 2.1% to 5%, by 2.2% to 5%, by 2.3% to 5%, by 2.4% to 5%, by 2.5% to 5%, by 2.6% to 5%, by 2.7% to 5%, by 2.8% to 5%, by 2.9% to 5%, by 3% to 5%, by 3.1% to 5%, by 3.2% to 5%, by 3.3% to 5%, by 3.4% to 5%, by 3.5% to 5%, by 3.6% to 5%, by 3.7% to 5%, by 3.8% to 5%, by 3.9% to 5%, by 4% to 5%, by 4.1% to 5%, by 4.2% to 5%, by 4.3% to 5%, by 4.4% to 5%, by 4.5% to 5%, by 4.6% to 5%, by 4.7% to 5%, by 4.8% to 5%, by 4.9% to 5%, by 0.5% to 4.5%, by 0.6% to 4.5%, by 0.7% to 4.5%, by 0.8% to 4.5%, by 0.9% to 4.5%, by 1% to 4.5%, by 1.1% to 4.5%, by 1.2% to 4.5%, by 1.3% to 4.5%, by 1.4% to 4.5%, by 1.5% to 4.5%, by 1.6% to 4.5%, by 1.7% to 4.5%, by 1.8% to 4.5%, by 1.9% to 4.5%, by 2% to 4.5%, by 2.1% to 4.5%, by 2.2% to 4.5%, by 2.3% to 4.5%, by 2.4% to 4.5%, by 2.5% to 4.5%, by 2.6% to 4.5%, by 2.7% to 4.5%, by 2.8% to 4.5%, by 2.9% to 4.5%, by 3% to 4.5%, by 3.1% to 4.5%, by 3.2% to 4.5%, by 3.3% to 4.5%, by 3.4% to 4.5%, by 3.5% to 4.5%, by 3.6% to 4.5%, by 3.7% to 4.5%, by 3.8% to 4.5%, by 3.9% to 4.5%, by 4% to 4.5%, by 4.1% to 4.5%, by 4.2% to 4.5%, by 4.3% to 4.5%, by 4.4% to 4.5%, by 0.5% to 4%, by 0.6% to 4%, by 0.7% to 4%, by 0.8% to 4%, by 0.9% to 4%, by 1% to 4%, by 1.1% to 4%, by 1.2% to 4%, by 1.3% to 4%, by 1.4% to 4%, by 1.5% to 4%, by 1.6% to 4%, by 1.7% to 4%, by 1.8% to 4%, by 1.9% to 4%, by 2% to 4%, by 2.1% to 4%, by 2.2% to 4%, by 2.3% to 4%, by 2.4% to 4%, by 2.5% to 4%, by 2.6% to 4%, by 2.7% to 4%, by 2.8% to 4%, by 2.9% to 4%, by 3% to 4%, by 3.1% to 4%, by 3.2% to 4%, by 3.3% to 4%, by 3.4% to 4%, by 3.5% to 4%, by 3.6% to 4%, by 3.7% to 4%, by 3.8% to 4%, by 3.9% to 4%, by 0.5% to 3.5%, by 0.6% to 3.5%, by 0.7% to 3.5%, by 0.8% to 3.5%, by 0.9% to 3.5%, by 1% to 3.5%, by 1.1% to 3.5%, by 1.2% to 3.5%, by 1.3% to 3.5%, by 1.4% to 3.5%, by 1.5% to 3.5%, by 1.6% to 3.5%, by 1.7% to 3.5%, by 1.8% to 3.5%, by 1.9% to 3.5%, by 2% to 3.5%, by 2.1% to 3.5%, by 2.2% to 3.5%, by 2.3% to 3.5%, by 2.4% to 3.5%, by 2.5% to 3.5%, by 2.6% to 3.5%, by 2.7% to 3.5%, by 2.8% to 3.5%, by 2.9% to 3.5%, by 3% to 3.5%, by 3.1% to 3.5%, by 3.2% to 3.5%, by 3.3% to 3.5%, by 3.4% to 3.5%, by 0.5% to 3%, by 0.6% to 3%, by 0.7% to 3%, by 0.8% to 3%, by 0.9% to 3%, by 1% to 3%, by 1.1% to 3%, by 1.2% to 3%, by 1.3% to 3%, by 1.4% to 3%, by 1.5% to 3%, by 1.6% to 3%, by 1.7% to 3%, by 1.8% to 3%, by 1.9% to 3%, by 2% to 3%, by 2.1% to 3%, by 2.2% to 3%, by 2.3% to 3%, by 2.4% to 3%, by 2.5% to 3%, by 2.6% to 3%, by 2.7% to 3%, by 2.8% to 3%, by 2.9% to 3%, by 0.5% to 2.5%, by 0.6% to 2.5%, by 0.7% to 2.5%, by 0.8% to 2.5%, by 0.9% to 2.5%, by 1% to 2.5%, by 1.1% to 2.5%, by 1.2% to 2.5%, by 1.3% to 2.5%, by 1.4% to 2.5%, by 1.5% to 2.5%, by 1.6% to 2.5%, by 1.7% to 2.5%, by 1.8% to 2.5%, by 1.9% to 2.5%, by 2% to 2.5%, by 2.1% to 2.5%, by 2.2% to 2.5%, by 2.3% to 2.5%, by 2.4% to 2.5%, by 0.5% to 2%, by 0.6% to 2%, by 0.7% to 2%, by 0.8% to 2%, by 0.9% to 2%, by 1% to 2%, by 1.1% to 2%, by 1.2% to 2%, by 1.3% to 2%, by 1.4% to 2%, by 1.5% to 2%, by 1.6% to 2%, by 1.7% to 2%, by 1.8% to 2%, by 1.9% to 2%, by 0.5% to 1.5%, by 0.6% to 1.5%, by 0.7% to 1.5%, by 0.8% to 1.5%, by 0.9% to 1.5%, by 1% to 1.5%, by 1.1% to 1.5%, by 1.2% to 1.5%, by 1.3% to 1.5%, by 1.4% to 1.5%, by 0.5% to 1%, by 0.6% to 1%, by 0.7% to 1%, by 0.8% to 1%, by 0.9% to 1% compared to a method not using the first alpha-amylase or using another first alpha-amylase.

In a preferred embodiment, the ethanol yield of the method using the first alpha-amylase and the composition comprising the second alpha-amylase and the protease is increased by 1% to 2% compared to a method not using the first alpha-amylase or using another alpha-amylase.

In one embodiment, the ethanol yield of the method using the first alpha-amylase and the composition comprising the second alpha-amylase and the protease is increased by 0.5%, by 0.6%, by 0.7%, by 0.8%, by 0.9%, by 1%, by 1.1%, by 1.2%, by 1.3%, by 1.4%, by 1.5%, by 1.6%, by 1.7%, by 1.8%, by 1.9%, by 2%, by 2.1%, by 2.2%, by 2.3%, by 2.4%, by 2.5%, by 2.6%, by 2.7%, by 2.8%, by 2.9%, by 3%, by 3.1%, by 3.2%, by 3.3%, by 3.4%, by 3.5%, by 3.6%, by 3.7%, by 3.8%, by 3.9%, by 4%, by 4.1%, by 4.2%, by 4.3%, by 4.4%, by 4.5%, by 4.6%, by 4.7%, by 4.8%, by 4.9%, by 5% compared to a method not using the first alpha-amylase or using another first alpha-amylase.

In a preferred embodiment, the ethanol yield of the method using the first alpha-amylase and the composition comprising the second alpha-amylase and the protease is increased by 1.4% compared to a method not using the first alpha-amylase or using another first alpha-amylase.

In one embodiment, the first alpha-amylase is present at an amount of 0.02% to 0.03%, the mixture is incubated at a temperature of 82° C. to 95° C. and a pH of about 4.2 to 5.0 and the ethanol yield of the method using the first alpha amylase and the composition comprising the second alpha-amylase and the protease is increased by about 1.4% compared to a method not using the first alpha-amylase or using another first alpha-amylase.

In one embodiment, the method of starch processing further comprises distilling the fermentation product to produce ethanol and whole stillage. In one embodiment, the whole stillage is processed to produce one or more of wet distiller's grains with solubles (WDGS) and dried distiller's grains with solubles (DDGS).

The term “distilling” refers to the process of separating the components of a liquid mixture by using selective boiling and condensation. The distillation of fermented products produces distilled alcohol and the remaining mixture of non-fermented solids and water after removal of alcohol from the fermented mash is referred to as “whole stillage”.

The terms “wet distiller's grains with solubles (WDGS)” and “dried distiller's grains with solubles (DDGS)” refers to the solid by-products of alcohol production. WDGS contain unfermented and fermented grain residues that are recovered after distillation, centrifugation, filtration and/or evaporation and can be made up by processed thin stillage and wet cake. DDGS are typically recovered from the WDGS or wet cake by evaporation, centrifugation and drying. The skilled person is aware of the uses of WDGS and DDGS, including the use as animal feed.

The skilled person is aware of methods of processing whole stillage. Whole stillage is processed by methods comprising centrifugation, evaporation, drying and thermal oxidation. Typically, whole stillage is separated into thin stillage (primarily liquids) and wet cake (primarily solids) by centrifugation.

The term “corn oil” refers to an oil that is produced by a starch processing method wherein the raw plant material is corn, by evaporation of the water from the thin stillage followed by centrifugation. Corn oil is used as animal feed or for the production of biofuel. The amount of corn oil being produced can be determined by using a flow meter to measure the amount of corn oil and comparing that to the amount of corn being ground (bushels of corn being ground).

In one embodiment, the corn oil extraction yield of the method using the first alpha-amylase and the composition comprising the second alpha-amylase and the protease is increased compared to a method not using the first alpha-amylase.

The “corn oil extraction yield” refers to the amount of corn oil produced using the methods of the present invention. Corn oil yield is defined as lb (0.45 kg) of corn oil per bushel (56 lb or 25.4 kg) of corn input. In another embodiment, the corn oil extraction yield of the method using the first alpha-amylase and the composition comprising the second alpha-amylase and the protease is increased by 4% to 12%, by 5% to 12%, by 6% to 12%, by 7% to 12%, by 8% to 12%, by 9% to 12%, by 10% to 12%, by 11% to 12%, by 4% to 11%, by 5% to 11%, by 6% to 11%, by 7% to 11%, by 8% to 11%, by 9% to 11%, by 10% to 11%, by 4% to 10%, by 5% to 10%, by 6% to 10%, by 7% to 10%, by 8% to 10%, by 9% to 10%, by 4% to 9%, by 5% to 9%, by 6% to 9%, by 7% to 9%, by 8% to 9%, by 4% to 8%, by 5% to 8%, by 6% to 8%, by 7% to 8%, by 4% to 7%, by 5% to 7%, by 6% to 7%, by 4% to 6%, by 5% to 6%, by 4% to 5% compared to a method not using the first alpha-amylase or using another first alpha-amylase.

In a preferred embodiment, the corn oil extraction yield in the method using the first alpha-amylase and the composition comprising the second alpha-amylase and the protease is increased by 7% to 9% compared to a method not using the first alpha-amylase or using another first alpha-amylase.

In another embodiment, the corn oil extraction yield in the method using the first alpha-amylase and the composition comprising the second alpha-amylase and the protease is increased by 4%; by 5%, by 6%, by 7%, by 8%, by 9%, by 10%, by 11% or by 12% compared to a method not using the first alpha-amylase or using another first alpha-amylase. In a preferred embodiment, the corn oil extraction yield in method using the first alpha-amylase and the composition comprising the second alpha-amylase and the protease is increased by about 8% compared to a method not using the first alpha-amylase or using another first alpha-amylase.

In one embodiment, the first alpha-amylase is present at an amount of 0.02% to 0.03%, the mixture is incubated at a temperature of 82° C. to 95° C. and a pH of about 4.2 to 5.0 and the corn oil extraction yield of the method using the first alpha amylase and the composition comprising the second alpha-amylase and the protease is increased by about 8% compared to a method not using the first alpha-amylase or using another first alpha-amylase.

### Composition Comprising the First Alpha-Amylase and Second Alpha-Amylase

In one embodiment a composition comprising a variant of a first alpha-amylase and a second alpha-amylase is disclosed.

The first alpha-amylase, the second alpha-amylase, and protease are defined in the in the preceding section.

In one embodiment a composition comprising a variant of a first alpha-amylase and a second alpha-amylase is disclosed. The variant of the first alpha-amylase has an amino acid sequence which is at least 80% identical to the amino acid sequence according to any one of SEQ ID NOs: 1, 3, 4, 5, 6 and 7 and which comprises at least one amino acid modification at an amino acid residue position number selected from the group consisting of: 23, 33, 181, 260, 272, 323, 349, 357, 407, and 408 or a combination thereof in the numbering of any one of SEQ ID Nos. 1, 3, 4, 5, 6 and 7. In a preferred embodiment, the at least one amino acid modification is an amino acid substitution selected from the group consisting of: 23E, 33E, 181E, 260D/E, 272D/E, 323E, 349P, 357E, 407E and 408E or a combination thereof in the numbering of any one of SEQ ID Nos. 1, 3, 4, 5, 6 and 7. In a preferred embodiment the variant of the first alpha-amylase comprises the amino acid modifications of: (a) 260D, or 357E, or 407E, 408E, or 23E, 33E, 181E, 260E, 272D, 323E, 349P, 357E, and 407E, or 23E, 260E, 272E, and 407E. In a preferred embodiment, the second alpha-amylase is an alpha-amylase from Geobacillus stearothermophilus or a variant thereof. In one embodiment the composition further comprises a protease and/or glucoamylase.

The following examples are provided for illustrative purposes. It is thus understood that the examples are not to be construed as limiting. The skilled person will clearly be able to envisage further modifications of the principles laid out herein.

### EXAMPLES

### Example 1: Viscosity Measurement

### Equipment:

Brookfield DV-II+ Viscometer with appropriate spindle, water bath, hot plate equipped with mixing for a 60° C. water bath during viscosity reading, 300 mL jars, 26% H2SO4, slurry samples.

### Method:

Briefly, the first alpha-amylase according to SEQ ID NO: 1 and a second alpha-amylase were added to a slurry of corn and water, and the suspension was thoroughly mixed with a whisk. The beaker was closed, and incubated at the target temperature with agitation for a specified incubation time. The reaction was quenched by acidification, the mixture split in two beakers, and the viscosity measured at 60° C.

1 mL 26% sulfuric acid was added for every 100 g of liquefied mash to stop starch hydrolysis by the α-amylase. Two beakers were prepared for each sample, and all samples equilibrated to target temperature and pH in the water bath. Viscometer was used according to manual with vane spindle #72 (if necessary, spindle #73).

All samples within a set or experiments used the same spindle and RPM, with a target % torque reading within the 10-90 range. One sample beaker was placed into the 60° C. water bath, spindle was submerged into the sample. Starting from the highest torque reading on the viscometer display, readings were taken every second for 60 readings.

### Example 2: Starch Processing Using a First and Second Alpha-Amylase at Varying Enzyme Doses and Temperatures

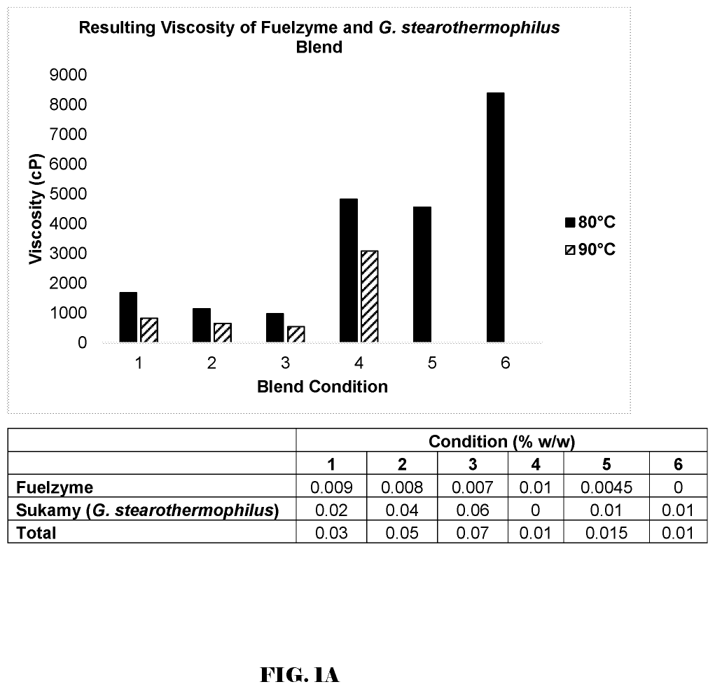

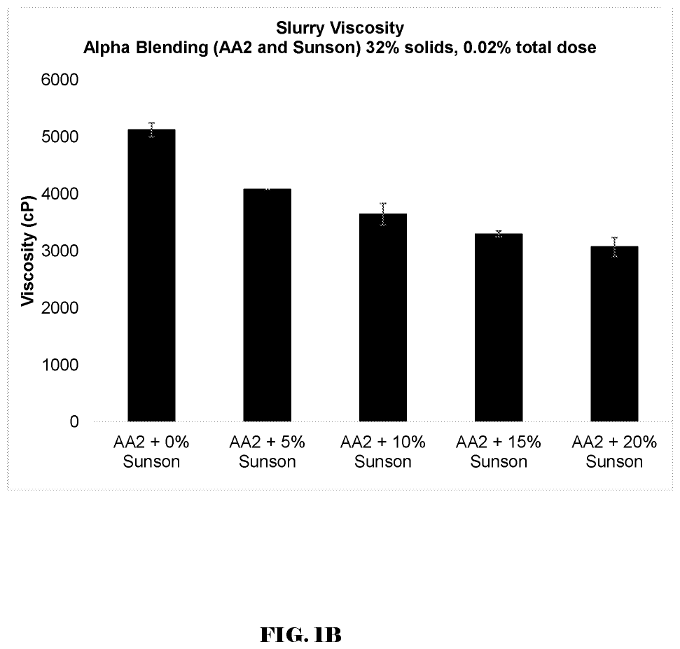

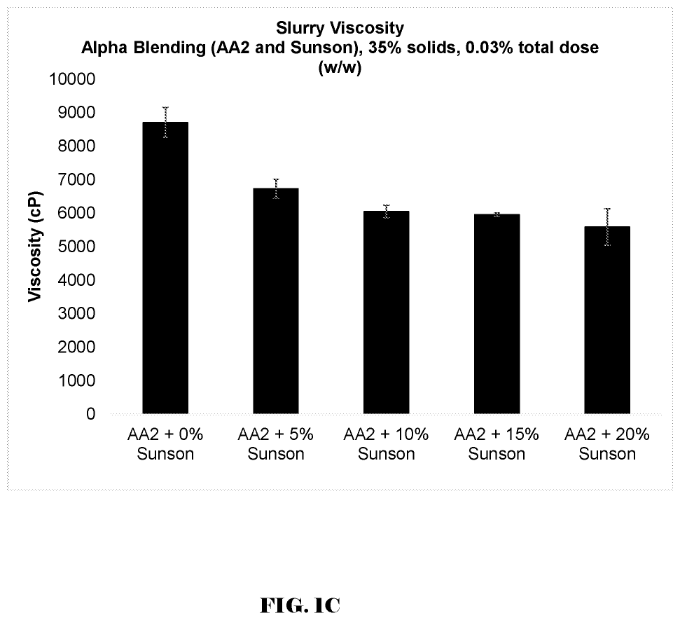

Experimental conditions can be found in Table 1. Briefly, the first alpha-amylase according to SEQ ID NO: 1 and the second alpha-amylase (SUKAMY HI) were added to a slurry comprising 30% solids at different doses. Enzyme doses can be found in FIG. 1a. The mixture was incubated at 80° C., 85° C. or 90° C. for 30 minutes before viscosity measurements were conducted as described above.

TABLE 1
Experimental conditions

| | |
|---|---|
| pH | 6.0 |
| Temperatures | 80° C., 85° C., 90° C. |
| Solids | 30% |
| Slurry Retention Time | 30 min |

Results: Viscosity was lower in slurries comprising the first alpha-amylase and the second alpha amylase compared to slurries only comprising the first alpha-amylase.

### Example 3: Starch Processing Using a First Alpha-Amylase and Second Alpha-Amylase at Different Ratios, Different Solids and Total Enzyme Dose

Experimental conditions can be found in Table 3. Briefly, the first alpha-amylase according to SEQ ID NO: 1 and the second alpha-amylase (Sunson HTAA180L) were added to a slurry containing either 32% (A) solids or 35% solids (B) at a total enzyme dose of 0.02% (A) or 0.03% (B) weight of enzyme per weight of raw plant material and inclusion rate of the second alpha-amylase was at 0%, 5%, 10%, 15% or 20%. Enzyme amounts are show in Table 4 for (A) and Table 5 for (B). The mixture was incubated at 80° C. for 15 minutes before viscosity measurements were conducted as described above.

Results: Viscosity was reduced in slurries comprising the first alpha-amylase and the second alpha-amylase compared to a slurry only comprising the first alpha-amylase in a dose-dependent manner, the viscosity decreased with an increased inclusion rate of the second alpha-amylase.

TABLE 3
Experimental conditions

| | |
|---|---|
| pH | 5.0 |
| Corn | In-house hammermilled corn (screen #5) |
| Enzymes | alpha-amylase according to SEQ ID NO: 1 |
| | Sunson HTAA180L (NX-H7-19435) |
| % Solid and total enzyme | A: 32% solids at 0.02% total enzyme dose |
| dose (% w/w dry basis) | B: 35% solids at 0.03% total enzyme dose |
| % Sunson HTAA 180L | 0%, 5%, 10%, 15%, 20% |
| addition (product | |
| weight %) | |
| System | Labomat (4° C./min ramp up) |
| Residence time | 15 min at 80° C. |
| Quenching method | H₂SO₄ injection to each Labomat beaker |
| Viscosity measurement | Brookfield vane spindle #73 |
| | Water bath at 60° C. |

TABLE 4
Enzyme doses at 32% solids and 0.02% total enzyme dose

| alpha-amylase according | Sunson | Inclusion |
|---|---|---|
| to SEQ ID NO: 1 | HTAA 180L | rate |
| 0.02% | 0% | 0% |
| 0.019% | 0.001% | 5% |
| 0.018% | 0.002% | 10% |
| 0.017% | 0.003% | 15% |
| 0.016% | 0.004% | 20% |

TABLE 5
Enzyme doses at 35% solids and 0.03% total enzyme dose

| alpha-amylase according | Sunson | Inclusion |
|---|---|---|
| to SEQ ID NO: 1 | HTAA 180L | rate |
| 0.03% | 0% | 0% |
| 0.0285% | 0.0015% | 5% |
| 0.027% | 0.003% | 10% |
| 0.0255% | 0.0045% | 15% |
| 0.024% | 0.006% | 20% |

### Example 4: Method of Starch Processing Comprising a First Alpha-Amylase and a Composition Comprising a Second Alpha-Amylase and a First Protease

Slurry, cook tube, and liquefaction temperatures were in the range of 85° C. to 105° C. and pH was in the range of pH 4.9 to pH 5.3. Viscosity out of slurry and liquefaction were in the acceptable range. Slurry and liquefaction DE were both in the appropriate range, which was 3-7 DE for the slurry and 8-13 DE for the liquefaction.

For the control conditions corn flour was mixed with hot process condensate/backset in conjunction with a Bacillus-based alpha-amylase enzyme (Novozymes SCDS) added to the slurry blender just before the slurry mix tank and also added after the jetting process just before the liquefaction tanks to form the liquefied mash. The total amount of the Bacillus-based alpha amylase added to the system was approximately 442 ml/min or 0.0505% weight of enzyme per weight of corn. The liquefaction tank provides residence time where the starch is hydrolysed into dextrins by the action of the alpha-amylase enzyme.

For the experimental conditions corn flour was mixed with hot process condensate/backset in conjunction with the first alpha-amylase according to SEQ ID NO: 1 added to the slurry blender just before the slurry mix tank and also added after the jetting process just before the liquefaction tanks to form the liquefied mash. The total amount of the first alpha amylase according to SEQ ID NO: 1 added to the system was approximately 270-400 ml/min or 0.027-0.040% weight of enzyme per weight of corn. During the experiment with the first alpha-amylase according to SEQ ID NO: 1 the alpha amylase enzyme dose to the liquefaction tank was eliminated at times. The liquefaction tank provides residence time where the starch is hydrolysed into dextrins by the action of the alpha-amylase enzyme.

In both control and experimental periods, a thermal stable protease was added to the slurry mix tank at a rate of 25-75 ml/min or 0.003-0.008% weight of enzyme per weight of corn as is.

The mash is later cooled to the optimum temperature for injection of additional enzymes and for yeast growth in fermentation.

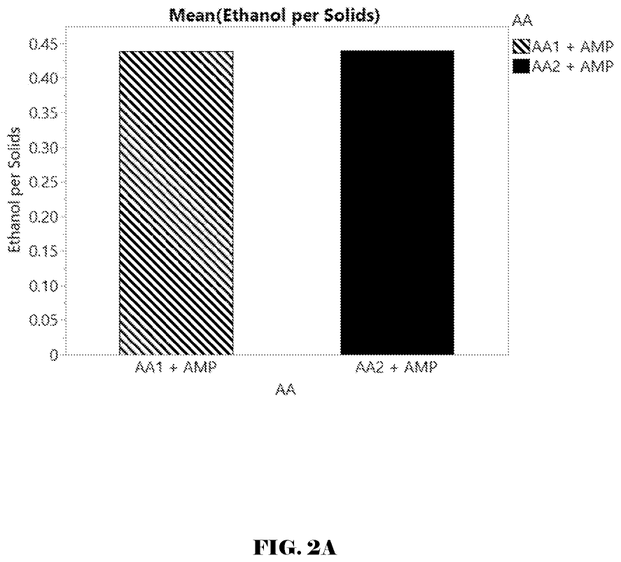

The experiment showed that the addition of the first alpha-amylase according to SEQ ID NO: 1 in conjunction with Novozymes Avantec® Amp, which contains a thermostable protease, an alpha amylase and potentially other enzymes resulted in higher ethanol and corn oil yields compared to the addition of Novozymes SCDS (bacillus based alpha amylase) in conjunction with Novozymes Avantec® Amp (see FIG. 2).

Ethanol yield is defined as gallons of ethanol produced per bushel of corn input. Fermentation ethanol yield was estimated by % concentration of ethanol determined by fermentation drop HPLC sample divided by the % solids concentration of the mash that fills the fermenter which is determined by drying a mash sample in a moisture balance. This is commonly referred to as fermentation ethanol yield ratio. Corn oil yield is defined as lb of corn oil per bushel of corn input and was estimated by lb of corn oil produced divided by bushels of corn ground in the same time period. A bushel of corn is defined as 56 lb or about 25.4 kg. The amount of ethanol is determined by using a flow meter to measure the amount of ethanol and corn oil being produced and comparing that to the amount of corn being ground (bushels of corn being ground).

Fermentations with the first alpha-amylase according to SEQ ID NO: 1 at the experiment conditions showed an increase in drop ethanol per corn solids of 1.38% with the use of the first alpha-amylase according to SEQ ID NO: 1 at a 0.027% wt./wt. dose. Corn oil extraction yield showed an increase of approximately 7.8% during the experiment period with the use of the first alpha-amylase according to SEQ ID NO: 1 at a 0.027% wt./wt. dose.

### Example 5: Starch Processing Using a First Alpha-Amylase and Second Alpha-Amylase at Different Ratios

Variants of the alpha-amylases according to SEQ ID NO: 1, 3, 4, 5, 6 and 7 were created as shown in Table 6 below.

TABLE 6
Alpha-amylase variants

| Sample Name | Parent sequence | Amino acid modifications |
|---|---|---|
| Variant 1 | SEQ ID NO: 3 | 260D |
| Variant 2 | SEQ ID NO: 3 | 357E |
| Variant 3 | SEQ ID NO: 3 | 408E |
| Variant 4 | SEQ ID NO: 3 | 23E, 33E, 181E, 260E, 272D, 323E, |
| | | 357E, 349P, 407E |
| Variant 5 | SEQ ID NO: 3 | 23E, 260E, 272E, 407E |
| Variant 6 | SEQ ID NO: 4 | 260D |
| Variant 7 | SEQ ID NO: 4 | 357E |
| Variant 8 | SEQ ID NO: 4 | 408E |
| Variant 9 | SEQ ID NO: 4 | 23E, 33E, 181E, 260E, 272D, 323E, |
| | | 357E, 349P, 407E |
| Variant 10 | SEQ ID NO: 4 | 23E, 260E, 272E, 407E |
| Variant 11 | SEQ ID NO: 5 | 260D |
| Variant 12 | SEQ ID NO: 5 | 357E |
| Variant 13 | SEQ ID NO: 5 | 408E |
| Variant 14 | SEQ ID NO: 5 | 23E, 33E, 181E, 260E, 272D, 323E, |
| | | 357E, 349P, 407E |
| Variant 15 | SEQ ID NO: 5 | 23E, 260E, 272E, 407E |
| Variant 16 | SEQ ID NO: 6 | 260D |
| Variant 17 | SEQ ID NO: 6 | 357E |
| Variant 18 | SEQ ID NO: 6 | 408E |
| Variant 19 | SEQ ID NO: 7 | 260D |
| Variant 20 | SEQ ID NO: 7 | 357E |
| Variant 21 | SEQ ID NO: 7 | 408E |
| Variant 22 | SEQ ID NO: 7 | 23E, 33E, 181E, 260E, 272D, 323E, |
| | | 357E, 349P, 407E |
| Variant 23 | SEQ ID NO: 7 | 23E, 260E, 272E, 407E |
| Variant 24 | SEQ ID NO: 1 | 407E |

Briefly, the first alpha-amylase (variant 4, 5, 7, 8, 12, 13, 17, 18 or 24) and the second alpha-amylase (Sunson HTAA180L) were added to a slurry containing 35% solids at an inclusion rate of the second alpha-amylase of 0%, 5%, 10%, 15% or 20%. Enzyme amounts are show in Tables 8 to 11. The mixture was incubated at 80° C. for 15 minutes before viscosity measurements were conducted as described above.

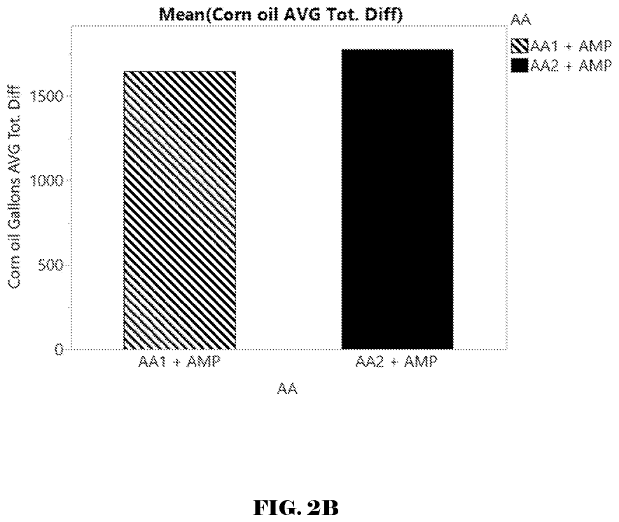

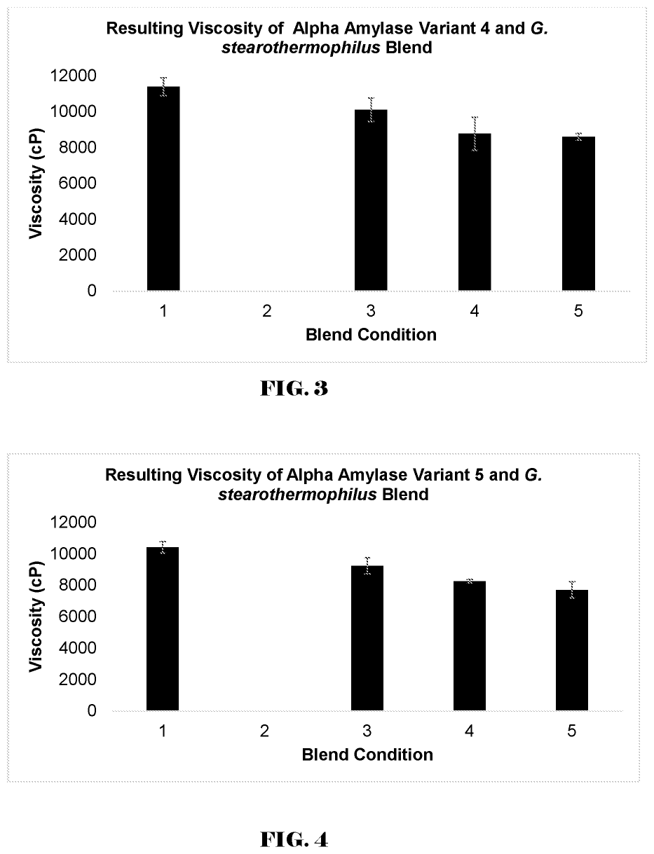

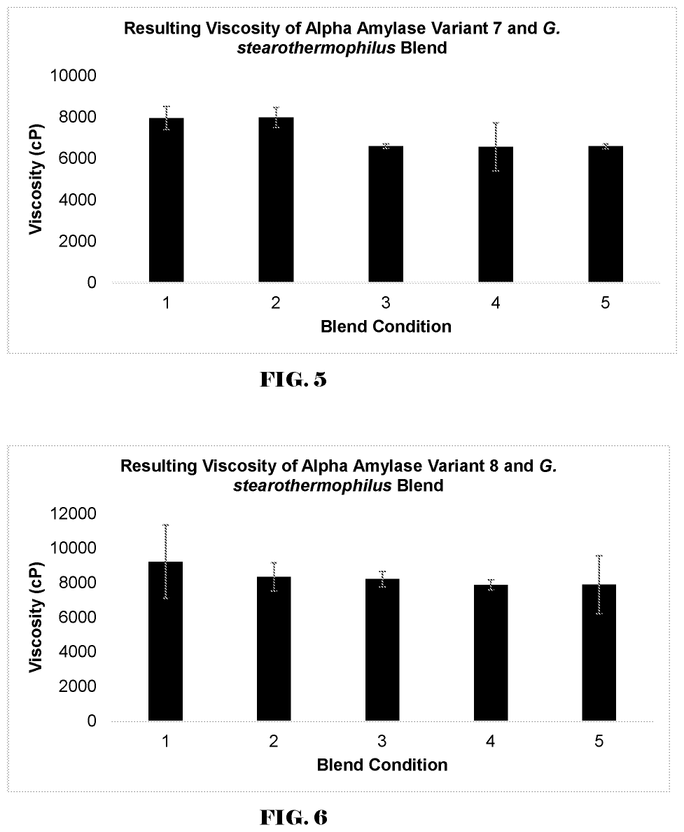

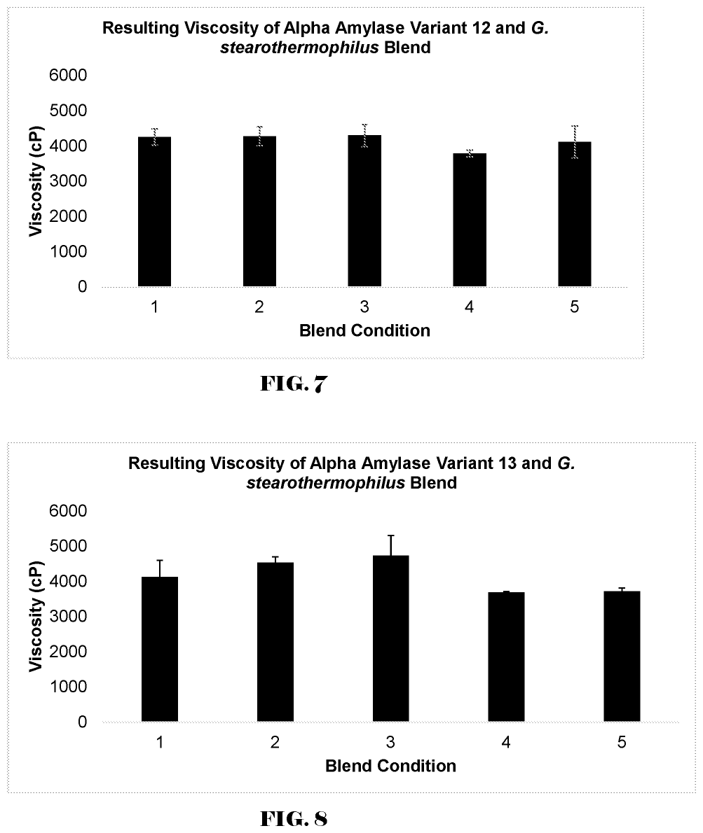

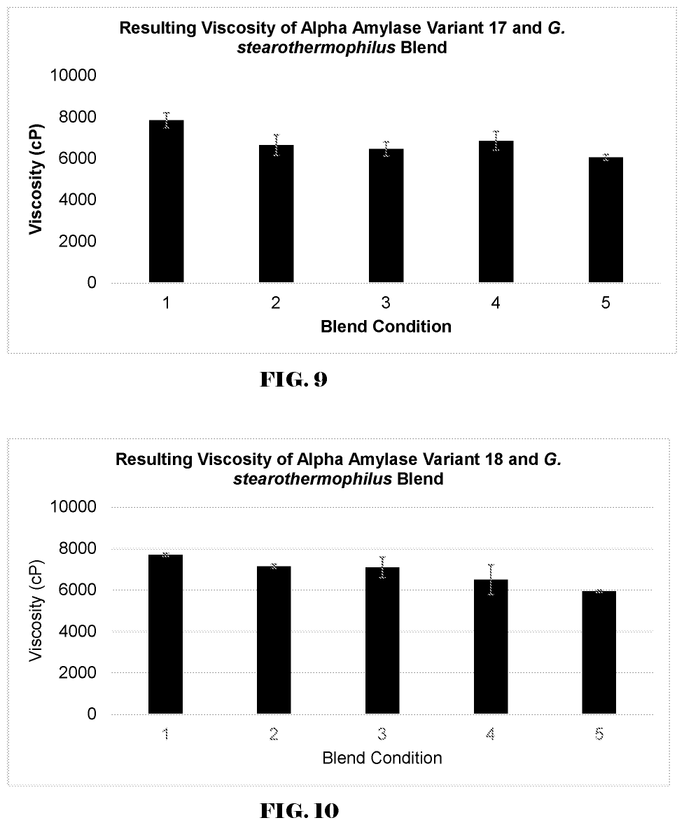

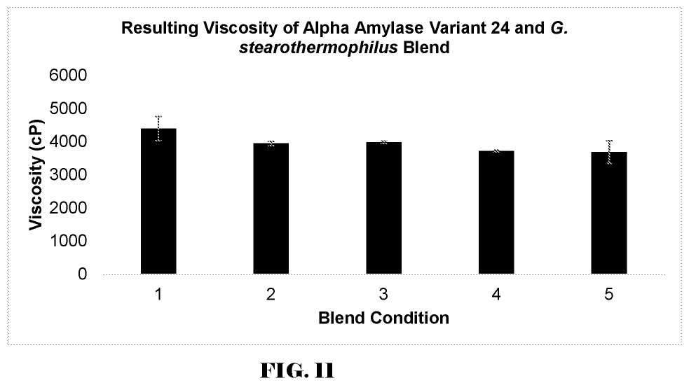

Results: Viscosity was reduced in slurries comprising the first alpha-amylase and the second alpha-amylase compared to a slurry only comprising the first alpha-amylase in a dose-dependent manner, the viscosity decreased with an increased inclusion rate of the second alpha-amylase (see FIGS. 3 to 11).

TABLE 7
Experimental conditions

| | |
|---|---|
| pH | 5.0 |
| Corn | In-house hammermilled corn (screen #5) |
| Enzymes | alpha-amylase variants 4, 5, 7, 8, 12, 13, |
| | 17, 18 or 24 |
| | Sunson HTAA180L (NX-H7-19435) |
| % Solid and total enzyme | 35% solids at 0.03% total enzyme dose |
| dose (% w/w dry basis) | |
| % Sunson HTAA 180L | 0%, 5%, 10%, 15%, 20% |
| addition (product | |
| weight %) | |
| System | Labomat (4° C./min ramp up) |
| Residence time | 15 min at 80° C. |
| Quenching method | H₂SO₄ injection to each Labomat beaker |
| Viscosity measurement | Brookfield vane spindle #73 |
| | Water bath at 60° C. |

TABLE 8
Conditions tested for Variants 4 and 5 with alpha-amylase from
G. stearothermophilus. Doses in % w/w of each product.

| | Condition (% w/w) | | | | |
|---|---|---|---|---|---|
| | 1 | 2 | 3 | 4 | 5 |
| Variant 4 or 5 | 0.02 | 0.0185 | 0.017 | 0.0155 | 0.014 |
| Sunson (G. | 0 | 0.0015 | 0.003 | 0.0045 | 0.006 |
| stearothermophilus) | | | | | |
| Total | 0.02 | 0.02 | 0.02 | 0.02 | 0.02 |

TABLE 9
Conditions tested for Variants 12 and 13 with alpha-amylase from
G. stearothermophilus. Doses in % w/w of each product.

| | Condition (% w/w) | | | | |
|---|---|---|---|---|---|
| | 1 | 2 | 3 | 4 | 5 |
| Variants 12 or 13 | 0.06 | 0.055 | 0.05 | 0.045 | 0.04 |
| Sunson (G. | 0 | 0.0015 | 0.003 | 0.0045 | 0.006 |
| stearothermophilus) | | | | | |
| Total | 0.06 | 0.0565 | 0.053 | 0.0495 | 0.046 |

TABLE 10
Conditions tested for Variants 7, 8, 17 and 18 with alpha-amylase
from G. stearothermophilus. Doses in % w/w of each product.

| | Condition (% w/w) | | | | |
|---|---|---|---|---|---|
| | 1 | 2 | 3 | 4 | 5 |
| Variants 7, 8, 17 or 18 | 0.033 | 0.03 | 0.027 | 0.024 | 0.02 |
| Sunson (G. | 0 | 0.0015 | 0.003 | 0.0045 | 0.006 |
| stearothermophilus) | | | | | |
| Total | 0.033 | 0.0315 | 0.03 | 0.0285 | 0.026 |

TABLE 11
Conditions tested for Variant 24 with alpha-amylase from
G. stearothermophilus. Doses in % w/w of each product.

| | Condition (% w/w) | | | | |
|---|---|---|---|---|---|
| | 1 | 2 | 3 | 4 | 5 |
| Variant 24 | 0.03 | 0.0285 | 0.027 | 0.0255 | 0.025 |
| Sunson (G. | 0 | 0.0015 | 0.003 | 0.0045 | 0.006 |
| stearothermophilus) | | | | | |
| Total | 0.03 | 0.03 | 0.03 | 0.03 | 0.03 |

1. A method of starch processing, comprising the steps of:
(a) providing a first alpha-amylase according to any one of SEQ ID NOs: 1, 3, 4, 5, 6 and 7 or a variant thereof comprising an amino acid sequence which is at least 80% identical to the amino acid sequence according to any one of SEQ ID NOs: 1, 3, 4, 5, 6 and 7;
(b) providing a second alpha-amylase;
(c) adding (a) and (b) to a slurry comprising a starch, thereby forming a mixture; and
(d) incubating said mixture.

2. The method of claim 1, wherein (a) comprises providing the variant of the first alpha-amylase, and wherein the variant of the first alpha-amylase comprises at least one amino acid modification at an amino acid residue position number selected from the group consisting of: 23, 33, 181, 260, 272, 323, 349, 357, 407, and 408 or a combination thereof in the numbering of any one of SEQ ID Nos. 1, 3, 4, 5, 6 and 7.

3. (canceled)

4. The method of claim 2, wherein the amino acid modification(s) is/are an amino acid substitution, and wherein the amino acid substitution is a conservative amino acid substitution.

5. The method of claim 2, wherein the at least one amino acid modification is an amino acid substitution selected from the group consisting of: 23E, 33E, 181E, 260D/E, 272D/E, 323E, 349P, 357E, 407E and 408E or a combination thereof in the numbering of any one of SEQ ID Nos. 1, 3, 4, 5, 6 and 7.

6. The method of claim 2, wherein the variant of the first alpha-amylase comprises the amino acid modifications of:
(a) 260D, or
(b) 357E, or
(c) 407E
(d) 408E, or
(e) 23E, 33E, 181E, 260E, 272D, 323E, 349P, 357E, and 407E, or
(f) 23E, 260E, 272E, and 407E
wherein the amino acid modifications are in the numbering of any one of SEQ ID Nos. 1, 3, 4, 5, 6 and 7.

7. The method of claim 1, wherein said mixture is incubated for a period of about 10 minutes to about 60 minutes.

8. The method of claim 1, wherein step (c) occurs during a liquefying stage and said mixture is incubated for a period of about 60 minutes to about 210 minutes during the liquefying stage.

9. (canceled)

10. The method of claim 1, wherein the method is performed at a temperature of about 62° C. to about 95° C.

11. The method of claim 1, wherein the method is performed at a pH of 4.3 to 6.5.

12. (canceled)

13. The method of claim 1, wherein the second alpha-amylase is an alpha-amylase from Geobacillus stearothermophilus or a variant thereof.

14. The method of claim 1, wherein the starch is derived from raw plant material and wherein the first alpha-amylase (a) is present in an amount of from about 0.001% to about 0.05% weight of enzyme by weight of raw plant material.

15.-17. (canceled)

18. The method of claim 1, wherein the viscosity of the slurry is reduced by at least 10% compared to a slurry not comprising the second alpha-amylase of (b).

19. The method of claim 1, wherein the slurry comprises 20% to 40% solids.

20. The method of claim 1, further comprising saccharifying and fermenting the slurry of step (c) to produce a fermentation product comprising alcohols.

21.-36. (canceled)

37. A composition comprising:
(a) a variant of a first alpha-amylase comprising an amino acid sequence which is at least 80% identical to the amino acid sequence according to any one of SEQ ID NOs: 1, 3, 4, 5, 6 and 7 and which comprises at least one amino acid modification at an amino acid residue position number selected from the group consisting of: 23, 33, 181, 260, 272, 323, 349, 357, 407, and 408 or a combination thereof in the numbering of any one of SEQ ID Nos. 1, 3, 4, 5, 6 and 7; and
(b) a second alpha-amylase.

38. (canceled)

39. The composition of claim 37, wherein the amino acid modification(s) is/are an amino acid substitution, and wherein the amino acid substitution is a conservative amino acid substitution.

40. (canceled)

41. (canceled)

42. The composition of claim 37, wherein the second alpha-amylase is an alpha-amylase from Geobacillus stearothermophilus or a variant thereof.

43. (canceled)

44. The method of claim 1, wherein (a) comprises providing the variant of the first alpha-amylase, and wherein the variant of the first alpha-amylase has at least 90% identity to any one of SEQ ID NOs: 1, 3, 4, 5, 6, and 7.

45. The method of claim 1, wherein (a) comprises providing the variant of the first alpha-amylase, and wherein the variant of the first alpha-amylase has at least 95% identity to any one of SEQ ID NOs: 1, 3, 4, 5, 6, and 7.

46. The composition of claim 37, wherein the variant of the first alpha-amylase is at least 90% identical to the amino acid sequence according to any one of SEQ ID NOs: 1, 3, 4, 5, 6 and 7.
